(19) United States
(12) Patent Application Publication
(10) Pub. No.: US 2020/0281729 A1
(43) Pub. Date: Sep. 10, 2020

(54) INSTRUMENTS AND METHODS FOR POSTERIOR SURGICAL APPROACH FOR SACROILIAC JOINT

(71) Applicant: Orthocision Inc., Folsom, CA (US)

(72) Inventors: Troy Schifano, Morgantown, WV (US); Steve Anderson, Folsom, CA (US); Teck-Mun Soo, Southfield, MI (US)

(21) Appl. No.: 16/810,823

(22) Filed: Mar. 5, 2020

Related U.S. Application Data
(60) Provisional application No. 62/813,773, filed on Mar. 5, 2019.

(51) Int. Cl. A61F 2/30; A61B 17/86; A61B 17/88; A61B 17/16

(52) U.S. Cl. CPC A61F 2/30988; A61B 17/8695; A61B 17/8875; A61B 17/1615; A61F 2002/30622; A61F 2002/3093; A61F 2002/30841; A61F 2002/30433; A61F 2002/30289; A61F 2002/30995

(57) ABSTRACT

Improved tools and methods of fusing human joints, such as the sacroiliac joint are disclosed. The methods use intra-articular joint fusion device(s) for fixing the articular surfaces relative to one another, The fusion implants may have fixation element(s) for engagement with bone tissue in the articular surfaces of the targeted joint. The present invention includes a novel washer device operable to stabilize instruments and the joint fusion implant.

### FIELD OF THE INVENTION

The present invention relates generally to medical devices and medical methods. More particularly, the present invention relates to musculoskeletal surgical methods and associated surgical tools for treatment of the sacroiliac joint.

### DISCUSSION OF THE BACKGROUND

Lower back pain is a common ailment among the population and results in both pain and suffering as well as loss of work time. Thus, approaches for the treatment of back pain can both relieve suffering as well as reduce employee down time. Thus, effective treatments for lower back pain have both economic benefits as well as the benefit of alleviating considerable suffering.

The sacroiliac joint is located in the lower back at the juncture of the ilium, the upper bone of the pelvis, and the sacrum at the base of the spine. While the sacroiliac joint has a limited range of motion, dysfunction of the joint has been identified. The joint is supported by a range of ligaments including, for example, the sacroiliac ligament at the base of the joint and the anterior sacroiliac ligament at the top of the joint.

The sacroiliac joint (SI joint) is increasingly being diagnosed as a common pain generator. That is, SI joint degenerative disease and instability are being diagnosed and treated more commonly. Sacroiliac pain may be caused by a disruption in the joint itself, a biomechanical problem like a muscle imbalance, trauma, an inflammatory condition like ankylosing spondylitis, or a degenerative problem as seen with post-lumbar fusion adjacent segment disorder. Other contributing factors include post pregnancy pain/instability, longer life span, and/or more active lifestyles. In addition, complex spine surgeries, such as for correction of sagittal plane deformity, often require iliac fixation to maintain correction in patients with a high pelvic incidence or high risk of lumbo-sacral hardware failure.

High energy pelvic ring injuries that involve disruption of the SI joint and/or displaced fractures of the sacrum present unique challenges to the orthopedic traumatologist. Some sacral fractures require solid posterior stabilization, which may be difficult to achieve with typical treatment methods. Furthermore, vertically unstable sacral fractures/SI joint disruptions have a relatively high incidence of neurovascular injury and may require unique stabilization. Typically, a spinal surgeon will be involved to perform lumbo-pelvic stabilization of these injuries to provide vertical stability of the injury. However, there may be significant soft tissue trauma associated with these injuries, making extensive surgical approaches of elevated risk in terms of infection and wound complications.

Immobilization of the SI joint can result in significant relief of lower back pain. Current techniques and instrumentation systems may require extensive surgical exposure and dissection. Moreover, such instrumentation systems are typically designed for other applications, and not to connect and stabilize the lumbar spine and pelvis. As a result, this can make the surgical times longer and more frustrating for surgeons and surgical staff. For example, traditional posterior iliac screws are often prominent because the posterior iliac crest is relatively subcutaneous. Yet, this sometimes makes hardware painful for the patient and at risk for pressure soreness following surgery.

Furthermore, the current techniques and instruments do not allow for a secure and consistent fusion construct. They may provide one or the other many times, but not both issues. This may lead to further SI joint instability and a failed surgery.

It is therefore desirable to provide new surgical methods and tools for treating damaged sacroiliac joints that securely and consistently fuse the joint.

### SUMMARY OF THE INVENTION

The present invention includes improved methods and devices for the immobilization or fusion of the Sacroiliac joint and apparatuses for facilitating the procedure. Immobilization may refer to mechanical holding and/or surgical fusion.

The present invention provides a system and surgical tools for introducing joint fusion implants that may perform the functions of mechanical fixation and stability, compression, and bony fusion. The present invention also relates to improved implant devices that may perform the functions of mechanical fixation and stability, compression, and bony fusion. The present invention also relates to methods of introducing fusion implants into a targeted joint using novel insertion tools. Specifically, with respect to some embodiments, an approach is described to address the SI joint and other joints through a posterior access approach while delivering fusion device that has fixation elements for grasping and holding bone tissue (e.g., by piercing, penetrating, or otherwise grasping) and fixing the bones of the SI joint relative to one another. The fixation elements may be in the form of helical anchors and/or other fixation elements. In some embodiments, the fixation elements are operable to provide for compression across the sacroiliac joint, and may be so used.

In some embodiments, the invention a novel instrument kit that includes the fusion device described herein, a cannula for exposing the sacroiliac joint from a posterior approach, a washer structure for insertion into the cannula prior to the insertion of the fusion device into the sacroiliac joint that includes a channel and a driver for the fusion device operable to be inserted into the sacroiliac joint, and an inserter operable to insert the joint fusion device through the washer channel into the sacroiliac joint and implant the fusion device at or into the articular surfaces of the ilium and sacrum. The instrument kit may include additional surgical instruments for establishing an incision over the posterior aspect of the sacroiliac joint, such as a drill, guide wire, dilators, and other devices. The instrument kit of the present invention may be utilized to address the SI joint through a posterior approach while delivering both a fusion device to the SI joint for fixing the sacrum and ilium.

It is therefore an object of the present invention to provide an improved approach for both mechanical holding and surgical fusion using novel joint fusion implants described herein. The joint fusion implants described herein may be introduced through a posterior approach to address the SI joint and may perform the functions of fixation, compression, and bony fusion, providing a secure fixation element for mechanical stability and a bony fusion element that allows for fusion between the sacrum and the ilium.

It is also an object of the present invention to provide an improved, combined approach for both mechanical holding and surgical fusion using novel fusion devices that may be introduced using a novel instrument kit through a posterior approach. It is also an object of the present invention to provide novel fusion implants that are capable of performing the functions of fixation and bony fusion, allowing for stable fusion of the SI joint through a single posterior approach. In some embodiments, the fusion implants of the present invention may also compress the targeted joint to bring the constituent bones together prior to fusion.

In some embodiments, the present invention relates to a fusion implant, comprising an elongate body adapted for placement in an intra-articular space between articular surfaces of a joint in general longitudinal alignment with a plane between the articular surfaces of the joint; and at least one primary fixation element for engagement with bone tissue of the bones in the joint. In some embodiments, the at least one primary fixation element is operable to penetrate at least one of the articular surfaces of the joint. In some embodiments, the fusion implant may include secondary fixation elements that are operable to engage with the bone tissue in the joint (e.g., one or more articular surfaces therein). In some examples, the secondary fixation elements may include protrusions, invaginations, fluting, or other surface features on the primary fixation element(s) and/or body of the fusion implant. In some embodiments, the fusion implant may further include a channel or space operable to hold a fusion-promoting material.

In some embodiments, the present invention relates to a medical instrument kit, including a joint fusion implant having a body and at least one fixation element attached to the body for engagement with bone tissue in one or more articular surfaces of a joint, a cannula for insertion into an incision (e.g., over a sacroiliac joint) in a human or animal, a deployable washer that has a diameter that fits within the cannula, the deployable washer having a central canal therein to allow the passage of the joint fusion device therethrough, a washer driver for inserting the washer into the targeted joint space and establishing the washer in the joint space with stable purchase, and an inserter for the joint fusion implant having a distal end that engages the head of the joint fusion device and is operable to advance the joint fusion device through the washer and into place in the targeted joint and engage the at least one fixation element with the bone tissue in the targeted joint.

The washer may include distal tangs for fixing the position of the washer in the targeted joint and provide stable purchase of the washer in the joint and stabilize the cannula and other instruments during the insertion of the joint fusion device. The washer may also include guidance tabs that may protrude from the washer through the proximal end of the cannula such that they are visible to the surgeon using the kit, and the guidance tabs may include markings thereon that indicate the depth of the inserter, as the inserter is passed through the channel in the washer. For example, at least one of the tabs may have a first proximal marking that, when aligned with a corresponding mark or structure on the inserter, indicates that the inserter has placed the fusion implant device at a position such that the distal end of the at least one lateral fixation element is positioned adjacent to and/or initial contact with the bone tissue in the targeted joint. At least one of the tabs may also include a second distal marking that, when aligned with a corresponding mark or structure on the inserter, indicates that the inserter has advanced the fusion implant device to the point that the at least one lateral fixation element is fully implanted into the bone tissue in the targeted joint. The guidance tab(s) may also be operable to break away from the washer at a predetermined connection point with the washer to enable removal of the guidance tabs from the washer placed in the targeted joint. The guidance tabs may be removed from the washer by applying stress at the predetermined connection point, which may be pre-formed or pre-treated to allow the applied stress to separate the guidance tab from the washer. In some embodiments, the predetermined connection point may be pre-formed to have a narrow cross-section that can be easily broken by shear stress. In other embodiments, the pre-treatment of the predetermined connection point may include etching and/or scoring of the material of the predetermined connection point, a thermal and/or chemical treatment at the predetermined connection point, an intervening layer of joint material between the guidance tab and the washer, or other technique for providing a least-resistance breaking point between the guidance tab and the washer. For example, shear stress may be applied in a perpendicular or oblique direction relative to the length of the guidance tabs, which may snap off of the washer at the predetermined connection point.

The distal end of the driver may be pressure fit onto the washer and the guidance tabs to provide a combined structure that can be utilized to drive the washer into the targeted joint and firmly establish the washer therein. The driver and washer may have complementary, nesting structure features that allow for a tight pressure fit between the washer and the driver. In some embodiments, the driver may have slots or cut-outs for accommodating the guidance tab(s) of the washer to establish a close fit between the guidance tab(s) and the driver. For example, the slots or cut-outs may run longitudinally along the outer circumference of the driver and have a complementary cross-sectional shape to that of the washer tabs. In other embodiments, the driver may have coupling structures on its outer surface (e.g., pegs) and the guidance tabs may have coupling slots into which the coupling structures may be engaged, such that the guidance tabs of the washer are reversibly fixed to the outer surface of the driver. The driver may also have structural features at its distal end that are operable to nest within complementary features of the washer. For example, the driver may have a distal collar have a smaller outer diameter than the other portions of the driver, and the collar may be operable to nest within the inner diameter of the washer.

The driver may have a length that is greater than the at least one washer tab, thereby allowing the driver to be impacted at its proximal end with a mallet or other driving tool to advance the washer into the targeted joint without damaging the washer tabs. The driver may have blunt or flat proximal end that can be impacted with the driving tool. The combination of the washer and the driver provides a combined tool that can drive the distal tangs of the washer into the targeted joint exposed by incision and establish a stable purchase before the insertion of the joint fusion implant. Once the washer is firmly established in the joint, the driver can be disengaged from the washer and removed. For example, the washer can be retracted from the cannula in a coaxial and linear manner without rotation, such that the washer is left in the targeted joint, and the washer tabs are left attached to the washer.

Once the washer is established in the targeted joint, the inserter may be engaged with the proximal end of the joint fusion device and then used to advance the joint fusion device through the cannula and into the targeted joint. The inserter may have a distal end configured to attach to the joint fusion device, e.g., having a bit-receiving recess (e.g., star, hex, or other functional shape) for receiving a complementary protrusion on the head of a joint fusion device. The inserter may be used to advance the joint fusion implant to the washer to place the joint fusion device adjacent and superficial to the targeted joint. A first proximal marking on a guidance tab of the washer may indicate to the medical personnel that the at least one fixation element of the joint fusion device is at the superficial aspect of the targeted joint when a structure of (e.g., the shoulder) or a marking on the inserter is aligned therewith. The medical personnel may then rotate the inserter using a driving tool to cause the at least one lateral fixation element to penetrate the bone tissue of the targeted joint. The medical personnel may continue rotating the inserter until the joint fusion device is fully implanted into the bone tissue. For example, the at least one lateral fixation element may be a helical element that penetrates and implants into the bone tissue (prepared cortical bone) of at least one bone in the target joint. The inserter may be rotated in the rotational direction of the spiral of the helical element to drive the helical element into the bone tissue. One or more guide tabs may include a second distal marker that indicates that the inserter advanced the fusion implant device to the point that the at least one lateral fixation element is fully inserted into the bone tissue in the targeted joint.

The kit may further include additional instruments to establish the cannula in the posterior incision, including a drill operable to be passed through the cannula into the incision and drilling a void through the connective tissues in the targeted joint, as well as an impactor for driving the washer into the joint by striking the driver; an electrically powered driver for the implant inserter for rotating the inserter at a controlled rate; a power or manual driver for the implant inserter for rotating the inserter at a controlled rate, thereby driving the joint fusion implant into the articular surfaces in the targeted joint; joint cutting instruments; dilators; guide wires; guide pins; guide pin assemblies; a rasp; a box chisel; adjustable arms for stabilizing the cannula and/or other working channels; and other tools that may be utilized in establishing incisions and access to a joint or bone tissue.

In some embodiments, the present invention relates to a joint fixation method including creating an incision proximal to the patient's targeted joint (e.g., the SI joint), dilating the incision, engaging an exposure device with the incision, inserting the washer into the targeted joint, and inserting and securing a joint fusing implant in bone tissue of at least one bone in the targeted joint.

In some embodiments, the present invention relates to a method for repairing a sacroiliac joint of a patient that includes creating a first incision in the patient's skin proximal to the patient's sacroiliac joint; dilating the incision; inserting a cannula into the first incision; creating a void in the sacroiliac joint; inserting a washer into the joint having tangs on a distal end thereof to engage the tissue in the articular space of the sacroiliac joint; and inserting a joint fusion implant into the sacroiliac joint through a channel in the washer, the joint fusion having at least one fixation element for engagement with bone tissue in the articular surfaces of the sacrum and the ilium in the sacroiliac joint.

In some embodiments, the present invention relates to a method including preparing a patient for surgery (e.g., positioning the patient in a prone position to provide the surgeon access to the SI joint, administering general or local anesthesia, and the like); locating the SI joint and an incision point for access to the SI joint (e.g., by blunt finger palpation); insertion of a pin or wire to create an incision; insertion of a dilator over the pin and impacting the dilator to dilate the incision to a width through which instruments may be passed; inserting a cannula over the dilator; securing the cannula in position over the incision (e.g., with fixing pins); removing the dilator; inserting a drill bit apparatus through the cannula, using the drill bit apparatus in the cannula to displace tissue in the SI joint thereby creating a void; removing the drill bit apparatus; coupling a washer and a washer driver, and inserting the coupled washer and driver into the cannula and advancing the coupled washer and driver until one or more tangs on a distal end of the washer are in contact with the tissue in the targeted joint; driving the washer into the tissue of the joint to establish a stable purchase of the washer, wherein the washer includes guidance tabs that protrude through the cannula and have position markers thereon to guide the insertion of a joint fusion implant; removing the driver from the washer and the cannula; engaging the joint fusion implant with an inserter and passing the joint fusion implant through the cannula until a designated structure of the inserter is aligned with a first position marker on the guidance tab, indicating that the joint fusion implant is positioned through a channel in the washer and is positioned proximal to the void in the patient's SI joint; rotating an inserter attached to said joint fusion implant to drive at least one fixation element into the bone tissue of the articular surfaces of the sacrum and ilium in the patient's SI joint until the designated structure of the inserter is aligned with a second position marker on the guidance tab, indicating that the joint fusion implant is sufficiently implanted into the sacrum and ilium; disengaging the inserter; removing the cannula; removing the guidance tabs from the washer; and closing the incision.

Additional objects of the invention will be apparent from the detailed descriptions and the claims herein.

### BRIEF DESCRIPTION OF THE DRAWINGS

FIG. 1 is an anterior view of the bony anatomy of the pelvis and sacrum.

FIG. 2 is a posterior view of the bony anatomy of the pelvis and sacrum.

FIG. 3 is a right lateral view of the bony anatomy of the pelvis and sacrum.

FIG. 4 is a left lateral view of the bony anatomy of the pelvis and sacrum.

FIG. 5 is a superior view of the bony anatomy of the pelvis and sacrum.

FIG. 6 is an inferior view of the bony anatomy of the pelvis and sacrum.

FIG. 7 is an oblique view of the right sacroiliac joint.

FIG. 8 is a perspective view of a joint probe.

FIG. 9 is an enlarged view of the joint probe in FIG. 8.

FIG. 10 is an end view of the joint probe in FIG. 8.

FIG. 11 is a perspective view of a guide pin.

FIG. 12 is an end view of the guide pin in FIG. 11.

FIG. 13 is a perspective view of a joint cutting instrument.

FIG. 14 is an enlarged view of the joint cutting instrument in FIG. 13.

FIG. 15 is an end view of the joint cutting instrument in FIG. 13.

FIG. 16 is a perspective view of a t-handle addition for a joint cutting instrument.

FIG. 17 is an end view of the t-handle addition in FIG. 16.

FIG. 18 is a perspective view of a joint cutting assembly.

FIG. 19 is an exploded, perspective view of the joint cutting assembly in FIG. 18.

FIG. 20 is a perspective view of a fixation pin insertion assembly.

FIG. is an exploded, perspective view of the fixation pin insertion assembly in FIG. 20.

FIG. 22 is a side view of a box chisel.

FIG. 23 is a side view of a rasp.

FIG. 24 is a perspective view of a bone graft impactor.

FIG. 25a is a perspective view of a fusion implant having helical fixation elements according to an embodiment of the present invention.

FIG. 25b is a perspective view of a fusion implant having helical fixation elements according to an embodiment of the present invention.

FIG. 25c is a top plan view of a fusion implant having helical fixation elements according to an embodiment of the present invention.

FIG. 25d is a bottom plan view of a fusion implant having helical fixation elements according to an embodiment of the present invention.

FIG. 26a is a perspective view of a fusion implant having helical fixation elements according to an embodiment of the present invention.

FIG. 26b is a perspective view of a fusion implant having helical fixation elements according to an embodiment of the present invention.

FIG. 26c is a bottom plan view of a fusion implant having helical fixation elements according to an embodiment of the present invention.

FIG. 27a is a perspective view of a fusion implant having helical fixation elements according to an embodiment of the present invention.

FIG. 27b is a side elevation view of a fusion implant having helical fixation elements according to an embodiment of the present invention.

FIG. 27c is a bottom plan view of a fusion implant having helical fixation elements according to an embodiment of the present invention.

FIG. 28a is a perspective view of a fusion implant having a helical fixation element according to an embodiment of the present invention.

FIG. 28b is a side elevation view of a fusion implant having a helical fixation element according to an embodiment of the present invention.

FIG. 28c is a bottom plan view of a fusion implant having a helical fixation element according to an embodiment of the present invention.

FIG. 29a is a perspective view of a surgical tool according to an embodiment of the present invention.

FIG. 29b is a perspective view of a surgical tool according to an embodiment of the present invention.

FIG. 29c is a bottom plan view of a surgical tool according to an embodiment of the present invention.

FIG. 30a is a perspective view of a surgical tool according to an embodiment of the present invention.

FIG. 30b is a side elevation view of a surgical tool according to an embodiment of the present invention.

FIG. 30c is a bottom plan view of a surgical tool according to an embodiment of the present invention.

FIG. 31a is a perspective view of a surgical tool according to an embodiment of the present invention.

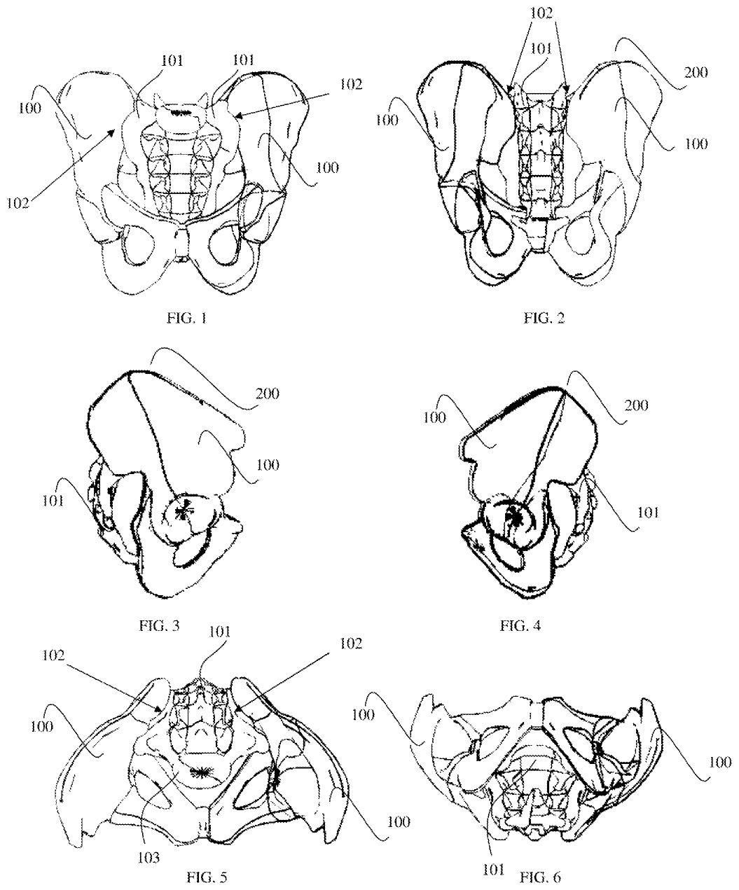

FIG. 3 lb is a perspective view of a surgical tool according to an embodiment of the present invention.

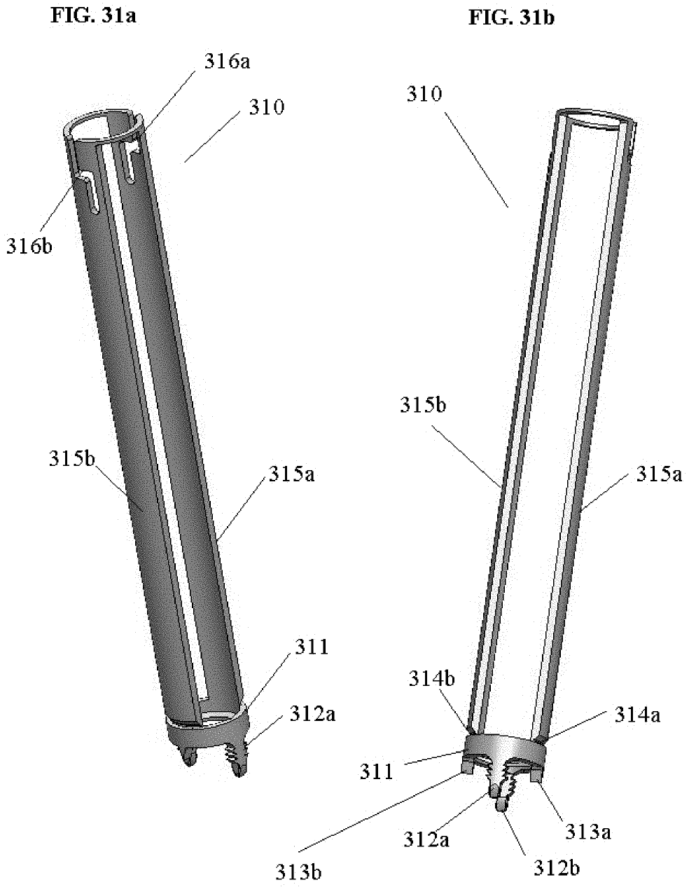

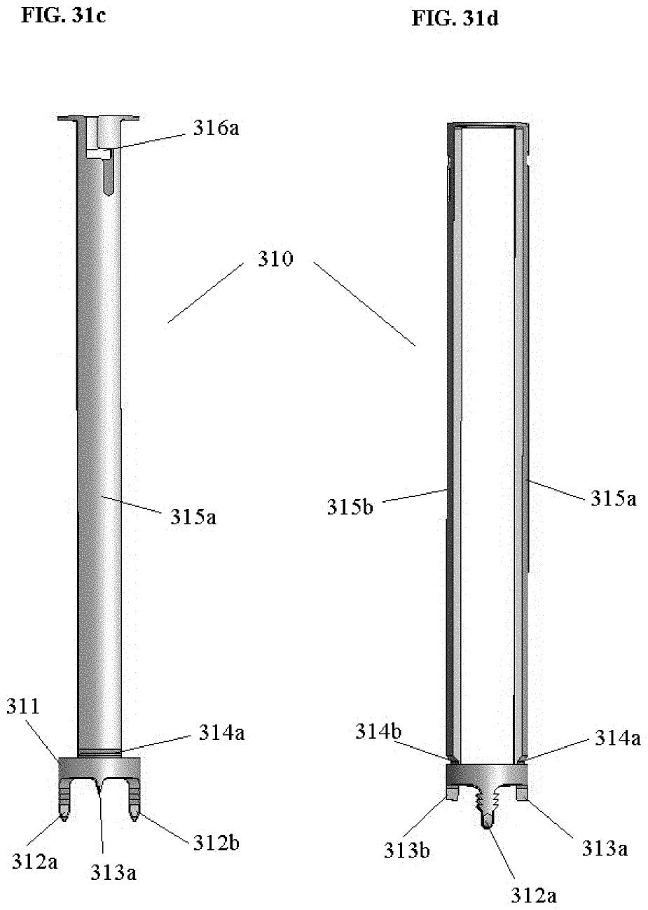

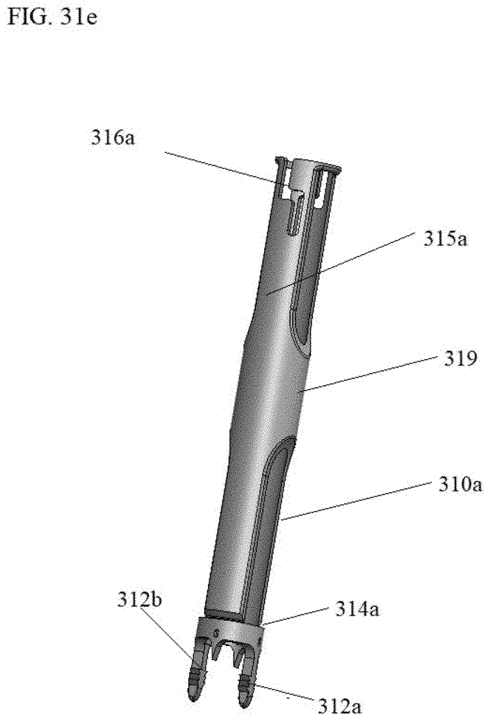

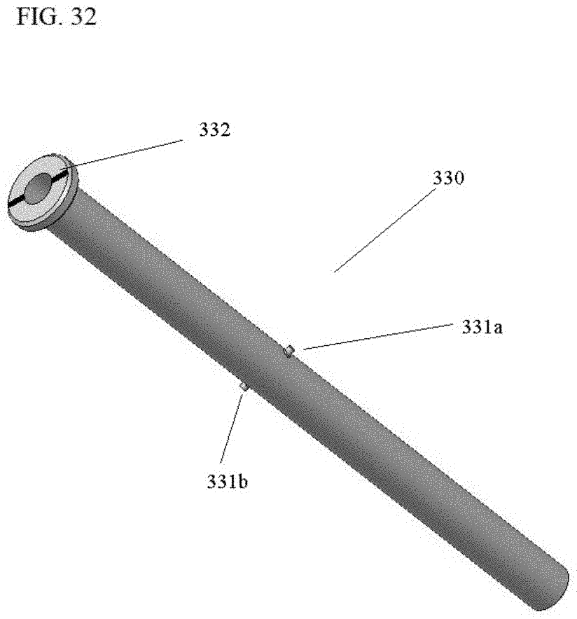

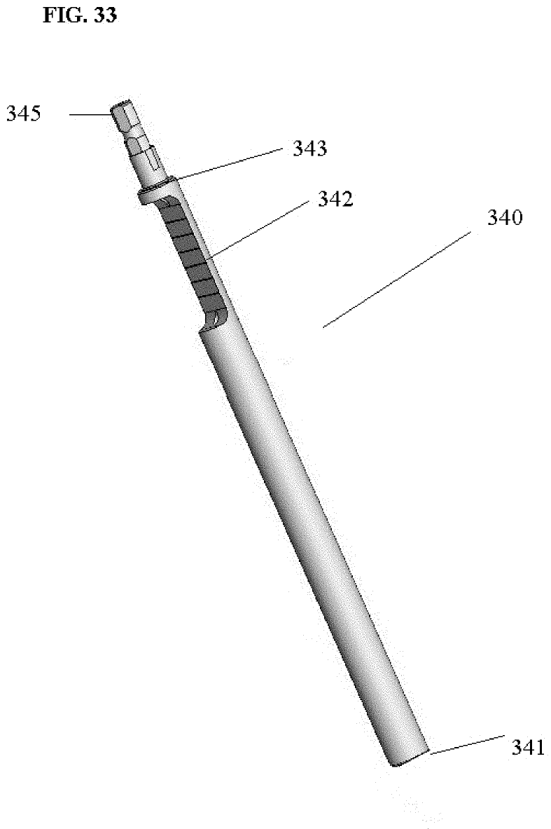

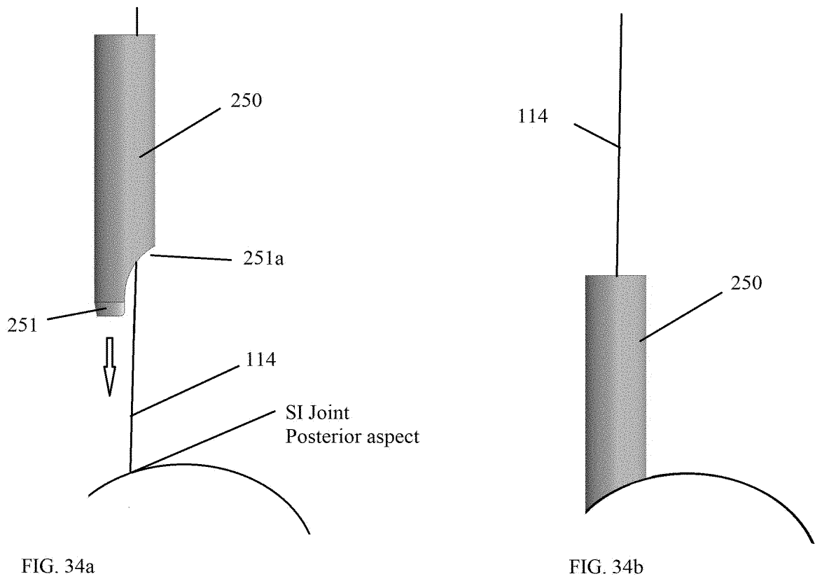

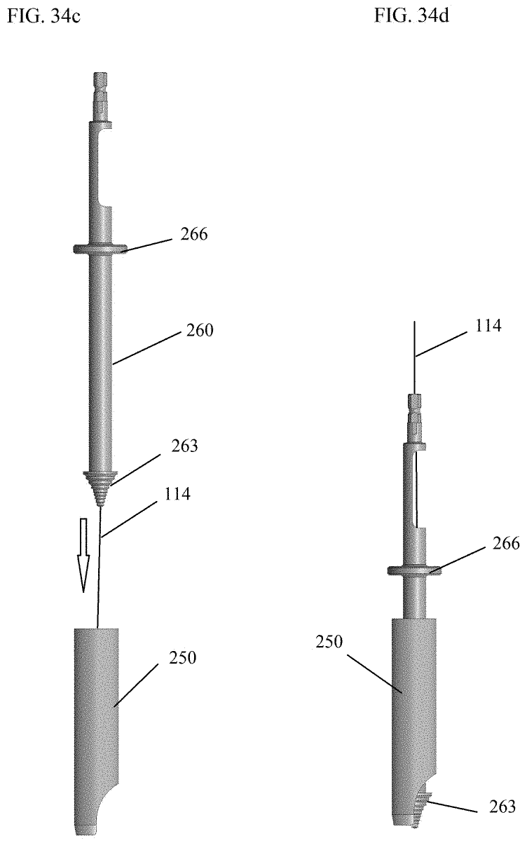

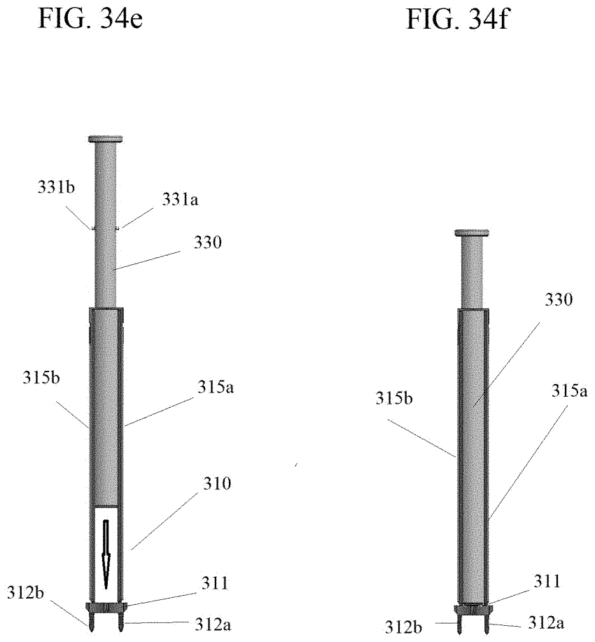

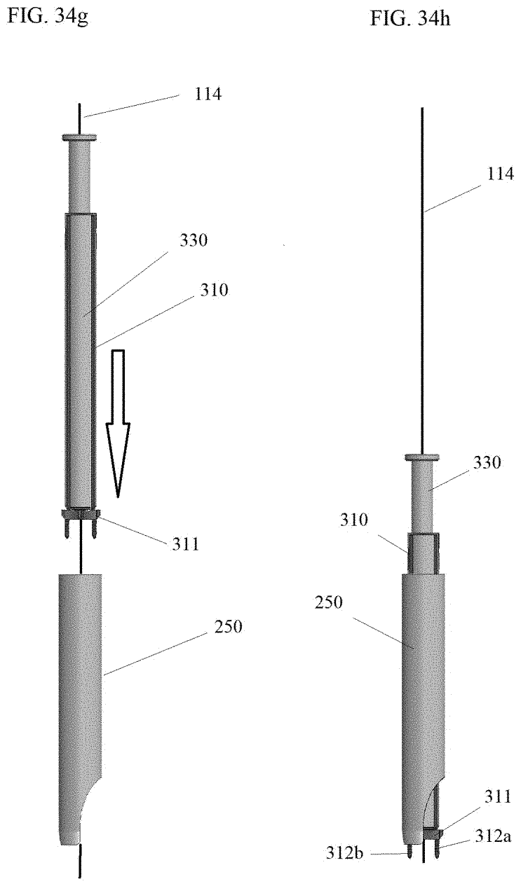

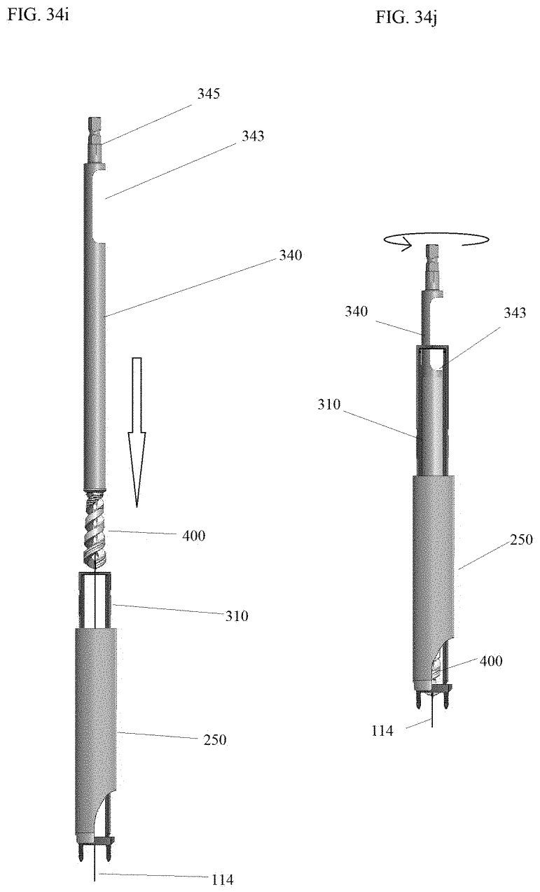

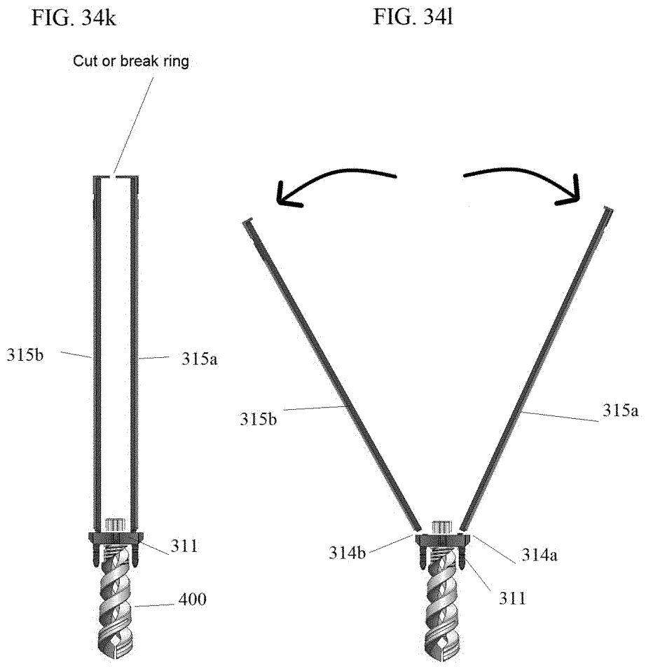

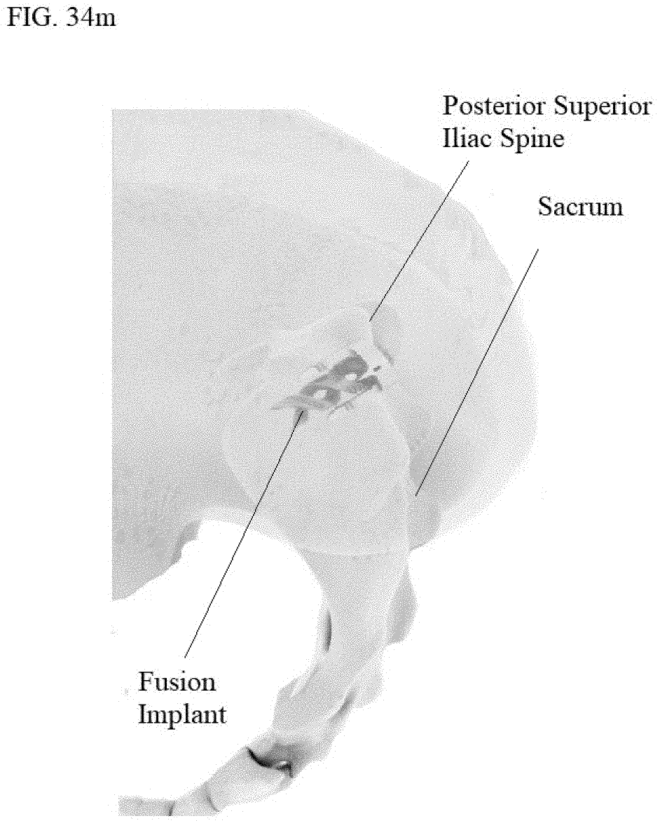

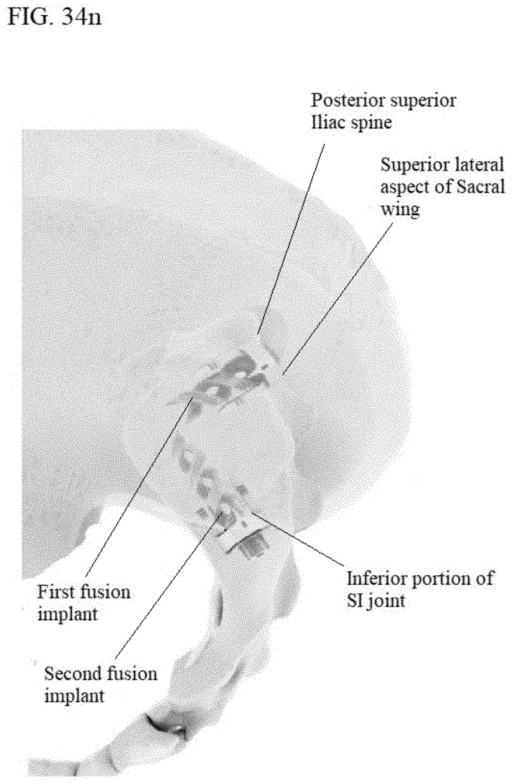

FIG. 31c is a side elevation view of a surgical tool according to an embodiment of the present invention.

FIG. 31d is a side elevation view of a surgical tool according to an embodiment of the present invention.

FIG. 31e is a perspective view of a surgical tool according to an embodiment of the present invention.

FIG. 32 is a perspective view of a surgical tool according to an embodiment of the present invention.

FIG. 33 is a perspective view of a surgical tool according to an embodiment of the present invention.

FIG. 34a illustrates a step in a surgical method according an embodiment of the present invention.

FIG. 34b illustrates a step in a surgical method according an embodiment of the present invention.

FIG. 34c illustrates a step in a surgical method according an embodiment of the present invention.

FIG. 34d illustrates a step in a surgical method according an embodiment of the present invention.

FIG. 34e illustrates a step in a surgical method according an embodiment of the present invention.

FIG. 34f illustrates a step in a surgical method according an embodiment of the present invention.

FIG. 34g illustrates a step in a surgical method according an embodiment of the present invention.

FIG. 34h illustrates a step in a surgical method according an embodiment of the present invention.

FIG. 34i illustrates a step in a surgical method according an embodiment of the present invention.

FIG. 34j illustrates a step in a surgical method according an embodiment of the present invention.

FIG. 34k illustrates a step in a surgical method according an embodiment of the present invention.

FIG. 34l illustrates a step in a surgical method according an embodiment of the present invention.

FIG. 34m illustrates a step in a surgical method according an embodiment of the present invention.

FIG. 34n illustrates a step in a surgical method according an embodiment of the present invention.

### DETAILED DESCRIPTION

Reference will now be made in detail to certain embodiments of the invention, examples of which are illustrated in the accompanying drawings. While the invention will be described in reference to these figures and certain implementations and examples of the embodiments, it will be understood that such implementations and examples are not intended to limit the invention. To the contrary, the invention is intended to cover alternatives, modifications, and equivalents that are included within the spirit and scope of the invention as defined by the claims. In the following disclosure, specific details are given to provide a thorough understanding of the invention. References to various features of the “present invention” throughout this document do not mean that all claimed embodiments or methods must include the referenced features. It will be apparent to one skilled in the art that the present invention may be practiced without these specific details or features.

Reference will be made to the exemplary illustrations in the accompanying drawings, and like reference characters may be used to designate like or corresponding parts throughout the several views of the drawings.

The present invention relates to novel fusion implants and surgical tools designed for repairing a damaged or injured joint in a human patient, particularly a sacroiliac joint, and methods for using such fusion implants and tools in procedures for repairing the damaged or injured joint. More specifically, the present invention pertains to a method for fixing and fusing a damaged joint using a fusion implant having fixation element(s) (e.g., integrally formed fixation elements). In some embodiments, the fusion implants of the present invention may be further used to compress the targeted joint. The fusion implant may include a gap, space, or channel operable to holding bone growth-promoting materials that provides for mechanical stability and promotes the formation of a contiguous piece of bone between two bones in the targeted joint (e.g., the sacrum and the ilium).

The fusion implants may be applied to the joint using a novel surgical tool kit without the need for additional patient positioning or secondary surgery. The novel surgical tool kit may include a joint fusion implant as described herein, a cannula for exposing the sacroiliac joint from a posterior approach, a washer structure for insertion into the cannula prior to the insertion of the fusion device into the sacroiliac joint that includes a channel and a driver for the fusion device operable to be inserted into the sacroiliac joint, and an inserter operable to insert the joint fusion device through the washer channel into the sacroiliac joint and implant the fusion device into the articular surfaces of the ilium and sacrum. The instrument kit may include additional surgical instruments for establishing an incision over the posterior aspect of the sacroiliac joint, such as a drill, guide wire, dilators, and other devices.

With respect to some embodiments, an approach is described to address the targeted joint through a posterior approach while delivering a fusion implant device that may fix and facilitate fusion of the joint. The fusion implant may also compress the targeted joint and/or deliver bone growth-promoting material (e.g., autologous bone, allograft, BMP, etc.). The fusion implant may be delivered to the joint, placed between bones in the targeted joint, and one or more fixation elements (e.g., integral fixation elements) of the fusion implant may be engaged with bone tissue in the articular surfaces of adjacent bones in the targeted joint to thereby fix the targeted joint. Engagement with the bone tissue may also compress the targeted joint.

The joint fusion implants of the present invention may include fixation elements that may be helical anchors positioned at or near a perimeter or circumference of the joint fusion implant. In such embodiments, the fixation elements are operable to be implanted in the tissue of the targeted joint, such as the cortical bone tissue, by rotating the joint fusion implant when it is positioned at the joint, adjacent to the bones of the joint. An implant inserter attached to the proximal end of the joint fusion implant may advance the joint fusion implant to the point that it is positioned at the bones of the targeted joint and may then be use to rotate the joint fusion implant to drive distal cutting ends of the fixation elements (e.g., helical anchors) of the joint fusion implant into the tissue of the joint, such as cortical tissue of the bone.

An exemplary surgical kit according to the present invention may include a washer structure for establishing stable purchase in the targeted joint prior to insertion of a joint fusion implant in order to stabilize a cannula through which the joint fusion implant is inserted into the joint and to prevent rotation or slippage of the cannula, implant insertion tool, or other instruments during the insertion and implantation of the joint fusion implant into the joint tissue (e.g., cortical bone tissue) of the targeted joint. Significant forces may be applied to the implant inserter in order to drive penetration of bone tissue by the bone-engaging (e.g., penetrating) ends of the fixation elements, which may cause torque to the joint and the attached surgical instrumentation that can cause destabilized movement of the surgical instruments. The establishment of the washer of the present invention in the targeted joint may prevent or reduce the risk of instrument rotation, slippage, or other displacement during the implantation of the joint fusion implant in the targeted joint. The washer and other instruments of the novel instrument kit of the present invention enables a minimally invasive surgical method for repairing a targeted joint (e.g., a sacroiliac joint) that results in a secure, consistent, and reliable fusion of the joint. The novel instrument kit enables the insertion of the joint fusion implant into the joint while avoiding damage to the soft and connective tissues in and around the joint by closely controlling the placement of the fusion implant.

### Relevant Anatomy Description

The surgical instruments, implants, and methods of the present invention are generally applicable to fixation and fusion procedures for planar, synarthrotic, and amphiarthrotic joints. The presently disclosed surgical instruments, implants, and methods are particularly applicable to such joints in the spine and pelvis. In particular, the surgical instruments, implants, and methods disclosed herein are applicable to fixation and fusion of the sacroiliac joint.

Referring to the drawings, FIG. 1 displays the bony anatomy of the sacrum and pelvis in a frontal, or anterior, view. The SI joint 102 is located between the ilium, or iliac wing, 100 and the sacrum 101 at the base of the pelvis 201. Additionally, the ridgeline of the articular process, the lateral ala 203 and the pedicle 104 of vertebrae 51 can be observed in this view. In FIG. 2, it can be seen that the SI joint 102 is not fully exposed for direct visualization from a rear, or posterior, viewpoint due to the angled and raised iliac crest 200 of the iliac wing 100. This angle provides a landmark for the entry point of the exposure device of the present invention at the posterior iliac crest. In FIG. 3 and FIG. 4, the prominence of the iliac crest 200 is displayed, along with the large surface area of the iliac wing 100, while the SI joint is fully enclosed between the iliac wing 100 and the sacrum 101 and occluded for direct visualization by the iliac wing 100. Again, the landmark of the posterior iliac crest can be seen. From a top down view, or superior view, the sacroiliac joint 102 can be fully observed between the iliac wing 100 and the sacrum 101, as shown in FIG. 5. Also shown in FIG. 5, the full sacrum 101 and specifically the vertebral body 103 of 51. The posterior superior iliac crest and the entry point of the 51 pedicle 104 can be observed in a direct line from one another.

In FIG. 6, it can be again observed that the SI joint is occluded from direct visualization due to the anatomy of the sacrum 101, the ilium 100 and the pelvis 201. Therefore, the only direct visualization of the SI joint can be achieved through an anterior, superior or posterior-oblique view of the sacrum and pelvis. Due to major organs being present in the pelvic-sacral cavity (colon, rectum, bladder, etc.), an anterior or superior approach to the bony anatomy and, specifically the SI joint, presents an unreasonable risk. Lateral approaches can be performed as described in Published U.S. Pat. No. 5,334,205 to Cain, entitled “Sacroiliac Joint Fixation Guide,” incorporated herein by reference and Published U.S. Pat. No. 8,221,428 to Trieu, entitled “Sacro-iliac joint implant system, method and instrument,” incorporated herein by reference. However, these techniques rely on non-direct confirmation methods such as navigation and fluoroscopy to determine accurate landmark and sacroiliac joint locations.

The present invention provides for novel surgical techniques, novel joint fusion implant, and instrument designs which allow for a direct visualization of the SI joint by utilizing a posterior-oblique access method to the anatomy as displayed in the oblique view of FIG. 7. In this drawing, the SI joint 102 can be clearly viewed between the right ilium 100 and sacrum 101. A corresponding joint may be exposed through the same approach on the left-hand side. Additionally, the anatomical landmark of the right posterior iliac crest and the corresponding access to the 51 pedicle 104 can be seen through this approach.

### Instruments

The present invention utilizes a novel instrument kit that may be used in a novel surgical method to introduce and secure a fusion implant into a targeted joint (e.g., an SI joint). The present invention also relates to novel fusion implants that may be implanted into the SI joint, for example, by the novel surgical tools and methods of the present invention. Exemplary instruments are described herein.

These surgical implements may be made of any suitable material, including medical grade plastics, metals, or alloys. In some embodiments, and without limitation, the tools are single use, in other embodiments the tools may be reused (and autoclaved, cleaned or otherwise suitably disinfected for further use). The tools may have various configurations, including those that differ from those depicted and specifically described herein.

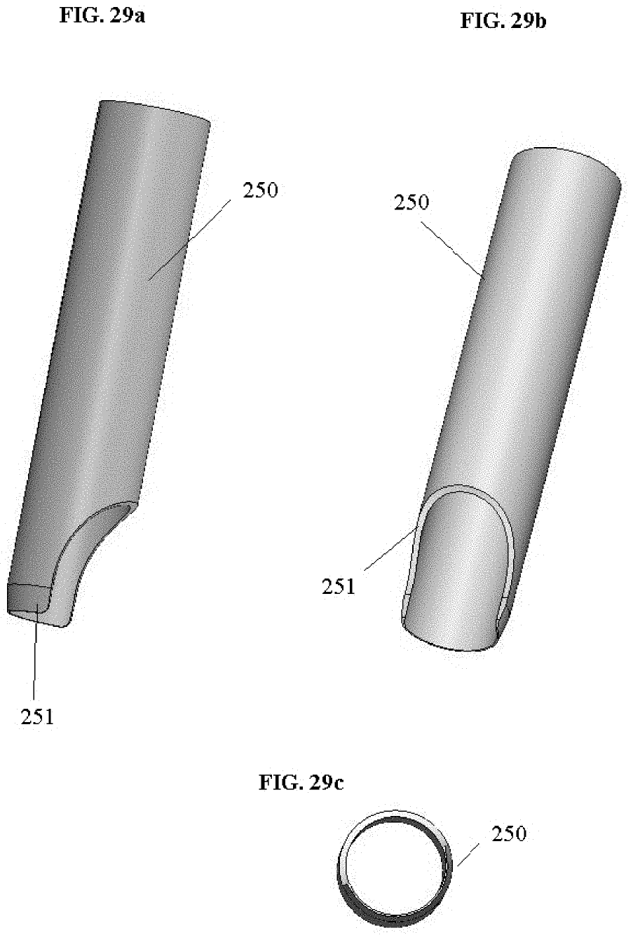

FIGS. 29a-29c show an exemplary cannula 250 that may be engaged with a posterior side of the targeted joint (e.g., sacroiliac joint) after an incision has been made thereover. The cannula 250 may have a hollow barrel therein for passing various surgical tools that may have a shape corresponding to (complementary to) the hollow barrel. The working channel may provide a guide for inserting the various surgical tools into the targeted joint, allowing precise surgical incisions, insertions of the joint fusion implant, etc. The barrel of the cannula 250 may have an interior perimeter shape that is complementary to and/or accommodative of the perimeter shape of the washer device 310 and the washer driver 330 of the present invention, allowing for a snug fit of the coupled washer device 310 and washer driver 330 in the interior barrel of the cannula 250. The matching of the perimeter shapes of the interior of the barrel and the coupled washer device 310 and washer driver 330 may allow the cannula to be proper oriented over the joint when the joint fusion implant is advanced into the targeted joint. For example, and without limitation, the exemplary cannula 250 may have a barrel having a substantially circular interior cross-section that may accommodate a joint fusion implant having circular cross-section (e.g., a helical implant) or a cross-section having a greatest diameter that is less than the diameter of the interior cross-section of the barrel.

The interior barrel of the cannula may also have guiding slots therein for properly aligning instruments (e.g., an implant inserter, an impactor, etc.). The guiding slots may engage notches or protrusions on the instruments such that the notches or protrusions slide along the guiding slots as the instrument or fusion implant is advanced through the barrel. Various implementations of the cannula of the present invention may have one or more guiding slots (e.g., 1, 2, 3, etc.) and they may be arranged in various spatial arrangements within the barrel.

The cannula may have an insertable end 251 for positioning within the incision over the targeted joint. The insertable end 251 may have a shape that is optimized for insertion into an incision over the targeted joint. The insertable end 251 may have a portion of its circumference removed to accommodate the bony protrusions and anatomy of the posterior pelvis around the sacroiliac joint. For example, the distal end of the cannula 251 may include a cutaway 251a to accommodate adjacent bony protuberances, e.g., the posterior iliac crest. The insertable end 251 may also have a tapered, rounded, and/or beveled edge that prevents or reduces damage to the soft and connective tissues in and around the incision of the targeted joint.

The cannula may have other additional features such as attachment points for handle(s) (e.g., for inserting handle extensions, etc.), or integrally formed handles as well. Additionally, the attachment points or handle(s) may also be attachable to a stabilizing structure (e.g., a table or surgical arm, retractor/stabilizing arms, etc.) to aid in preventing movement of the exposure device or surgical implements engaged therewith during surgical procedure. It is to be appreciated that the above description of the exposure tool does not limit the present invention, and other features are contemplated in and within the scope of the present invention.

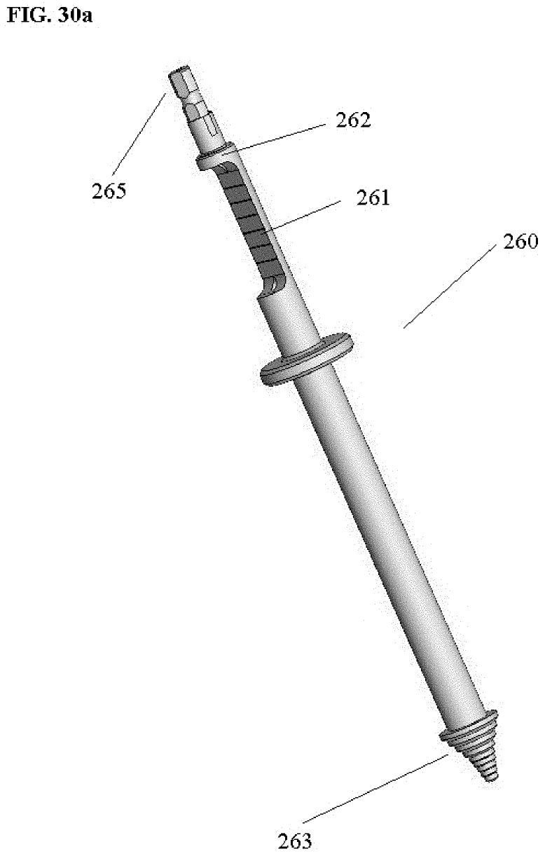

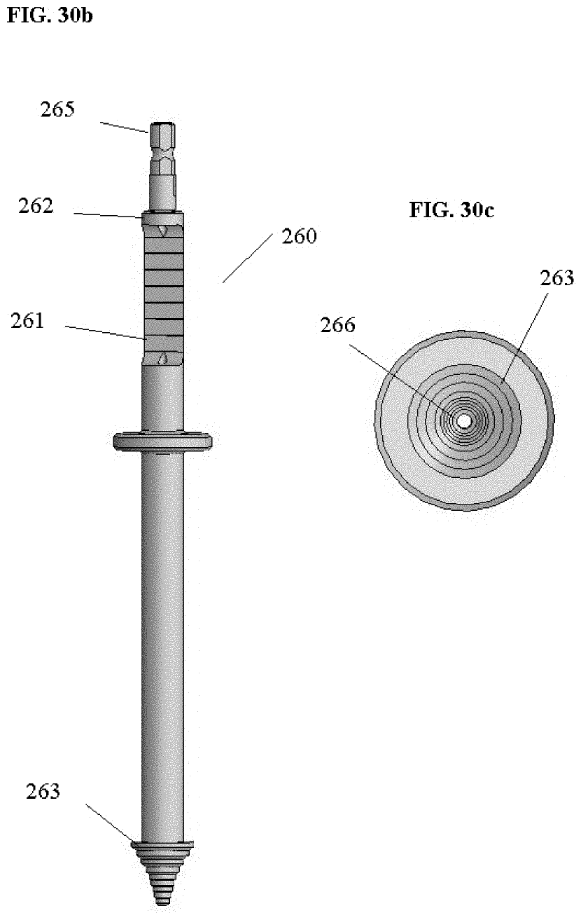

FIGS. 30a-30c show an exemplary drill 260 for bone preparation for fusion implant insertion. The drill 260 may be used for creating a void in the targeted joint for later insertion of a joint fusion implant. The drill 260 may have a recessed portion 261 along its proximal shaft that allows the medical personnel to monitor the depth to which the drill 260 penetrates the targeted joint. The shoulder 262 may be used as a marker used in conjunction with the cannula 250. The shoulder may have a pre-determined marking thereon that when aligned with the upper rim of the cannula 250 indicates that the drill has been advanced to desired depth in the targeted joint to provide a sufficient void for insertion of a joint fusion implant.

The drill 260 may have a threaded or stepped blade portion 263 with numerous concentric blade steps in order to provide a hole with the desired size void cut in the targeted anatomy. The blade step 263a with the widest diameter may have a diameter that is equal to or substantially equal to the outer diameter of the washer to allow for effective placement of the washer, nesting in the widest part of the void created by the drill in the targeted joint.

The drill 260 may also be designed to be attached to power instruments, a hand drill or a handle. Without limiting the invention, the exemplary drill 260 may have a Jacob's chuck connection 265 so that it may be attached to a powered drill for quick preparation. Additionally, the drill may have a central channel 266 running down its length that may allow a guide wire or pin to be inserted there through into the incision.

FIGS. 31a-31e, show an exemplary embodiment of a washer device 310 for insertion into a targeted joint (e.g., a sacroiliac joint) is shown. The washer device 310 includes a washer ring 311 at its distal end that may include lateral tangs 312a and 312b and one or more protrusions 313a and 313b for penetrating tissues in the targeted joint and establishing a stable purchase of the washer device 310 before a joint fusion implant is inserted and implanted into the targeted joint. The washer device 310 may further include guidance tabs 315a and 315b that connect to the washer ring 311 at connection points 314a and 314b. The guidance tabs 315a and 315b may be operable to be engaged with a washer driver 330 in order to couple the washer device 310 and the washer driver 330 together. The guidance tab may include coupling channels 316a and 316b that may be engaged with coupling pegs 331a and 331b of the washer driver 330. The guidance tabs 315a and 315b may be operable to be removed from the washer ring 311 at connection points 314a and 314b. For example, the material of the guidance tabs 315a and 315b may be sufficiently thin and/or pre-treated (e.g., scored, thermally treated, chemically treated, or otherwise prepared) such that the guidance tabs may be snapped or broken off at the connection points 314a and 314b such that they can be removed from the washer ring 311 and the incision over the targeted joint. In other examples, the guidance tabs 315a and 315b can be removed by other techniques, e.g., by a shearing or cutting tool.

The lateral tangs 312a and 312b may be operable to penetrate tissue in the targeted joint by being driven into the joint, e.g., by impaction. The lateral tangs may include features that provide for structures that facilitate firm lodging in the tissue (e.g., connective tissues, cartilage, prepared bone surface, etc.). Such feature may include serrations 313c along the surface of the lateral tangs 312a and 312b. Such serrations 313c may have a retrograde angle directed toward the proximal end of the washer 310 to prevent dislodgement of the washer 311 from the targeted joint. In some examples, the lateral tangs 312a and 312b may be arranged such that they are diametrically or substantially diametrically positioned on washer ring 311 such that they can be driven between two adjacent articular surfaces of bones in the target joint, allowing penetration of the joint by the tangs 312a and 312b. In other examples, the washer 310 may include other arrangements of tangs, such as a single tang, or multiple tangs arranged in various positions on the washer ring. The one or more protrusions 313a and 313b may aid in establishing a stable purchase of the washer 310 in the targeted joint, e.g., providing penetration of tissues at edges of or adjacent to the targeted joint.

FIG. 31e shows a further embodiment of a washer of the present invention, washer device 310a. Washer device 310a shares most of the same features with the other examples of the washer device, but has the added feature of bridges 319 between the guidance tabs 315a and 315b. The bridges 319 may additional structural stability to the washer device 310a. To remove the guidance tabs from washer device 310a, both guidance tabs may be pushed orthogonally to the washer ring 311 in the same direction and broken away at connection points 314a and 314b.

As shown in FIG. 32, the instrument kit of the present invention may also include a washer driver 330, which is operable to be coupled to the washer 310 to facilitate the insertion and penetration of the washer 310 into the targeted joint. In some examples, the driver 330 may have an external diameter that may fit within the inner diameter of the guidance tabs 315a and 315b of the washer 310 such that the guidance tabs 315a and 315b can be tightly fit over the washer driver 330. In such examples, the washer driver may also include coupling pegs 331a and 331b which may be interlocked with coupling channels 316a and 316b of the guidance tabs 315a and 315b. The coupling pegs 331a and 331b may be slotted into the coupling channels 316a and 316b to reversibly fix the washer device 310 and the washer driver 330 together. In other embodiments, the washer driver and washer may have complementary, nesting structure features that allow for a tight pressure fit between the washer device and the washer driver. For example, the driver may have slots or cut-outs for accommodating the guidance tab(s) of the washer device to establish a close fit between the guidance tab(s) and the driver. Such slots or cut-outs may run longitudinally along the outer circumference of the driver and have a complementary cross-sectional shape to that of the washer tabs. The washer driver may also having additional structural features at its distal end that are operable to engage with the washer device. For example, the distal end of the washer driver may have a distal collar have a smaller outer diameter than the other portions of the driver, and the collar may be operable to nest within the inner diameter of the washer ring.

The washer driver 330 may have a length that is greater than the guidance tabs 315a and 315b, thereby allowing the washer driver 330 to be impacted at its proximal end with a mallet or other driving tool to advance the washer device 310 into the targeted joint without damaging the guidance tabs 315a and 315b. The washer driver 330 may have blunt or flat proximal end 332 that can be impacted with a driving tool such as a mallet. The washer driver 330 may be operable to be driven through the cannula to in turn drive the lateral tangs 312a and 312b of the washer ring 311 into the targeted joint.

As shown in FIG. 33, the instrument kit may also include an implant inserter 340 operable to engage with a joint fusion implant and insert and implant the joint fusion implant into the targeted joint. Once the washer device 310 is established in the targeted joint, the implant inserter 340 may be engaged with the proximal end of a joint fusion implant and then used to advance the joint fusion implant through the cannula 250 and into the targeted joint. The implant inserter 340 may have a distal end 341 configured to attach to the joint fusion device, e.g., having a bit-receiving recess (e.g., star, hex, or other functional shape) for receiving a complementary head of a joint fusion device. The washer inserter 340 may have a mechanism for tightening the engagement of the bit recess with the head of the joint fusion implant. For example, an actuator operable by the medical personnel may control a spring-operated element, e.g., in the bit recess that reversibly applies pressure to the head of the joint fusion implant to maintain the joint head in the bit recess.

The implant inserter 340 may have a recessed portion 342 along its proximal shaft having a cutaway 343 that allows the medical personnel to monitor the depth to which the implant inserter 340 penetrates the targeted joint (e.g., the distal end of the cutaway can be used as a marker). The cutaway 343 may act as a marker to be used in conjunction with the markers on the guidance tabs 315a and 315b to determine when the implant inserter 340 has reached the desired depth to deliver a joint fusion implant into the targeted joint. For example, at least one washer tab may be marked to indicate the point to which the distal end of the cutaway 343 of the implant inserter should be advanced to completely insert he fusion implant into the targeted joint. In other examples, at least one washer tab may include two marks, a proximal mark to indicate the point to which the distal end of the cutaway 343 should be advance to place the distal end of the fusion implant at superficial aspect of the bones in the joint, and a distal mark to indicate the point to which the shoulder 343 of the implant inserter should be advanced to completely insert he fusion implant into the targeted joint.

The inserter 340 may be used to advance the joint fusion device to the washer ring 311 to place the joint fusion device adjacent and superficial to the targeted joint. The first proximal position marker on a guidance tab 312a of the washer device 310 may indicate to the medical personnel that the joint fusion device is at the superficial aspect of the targeted joint when the shoulder 343 of the implant inserter 340 is aligned therewith. The medical personnel may then rotate the implant inserter 340 using a driving tool to cause the at least one lateral fixation element of the joint fusion implant to penetrate the bone tissue of the targeted joint. The implant inserter 340 may have a Jacob's chuck connection 435 so that it may be attached to a driving tool. The medical personnel may continue rotating the implant inserter 340 until the shoulder 343 is aligned with the second distal position marker on a guidance tab 312a of the washer device 310, which indicates that the joint fusion implant is fully implanted into the bone tissue. Various additional instruments may be included in such a set, including a joint cutting instrument (e.g., dilator), guide pins, guide pin assemblies, a rasp, a box chisel, an inserter, an impactor, and other instruments. Each of such tools may correspond to the exposure devices described herein. For example, the joint cutting instrument, the rasp, the box chisel, and the impactor each may have a shape that is complementary to the barrel of the cannula, allowing each instrument to be inserted into the barrel and in the proper orientation, without room to deviate from the path of the barrel.

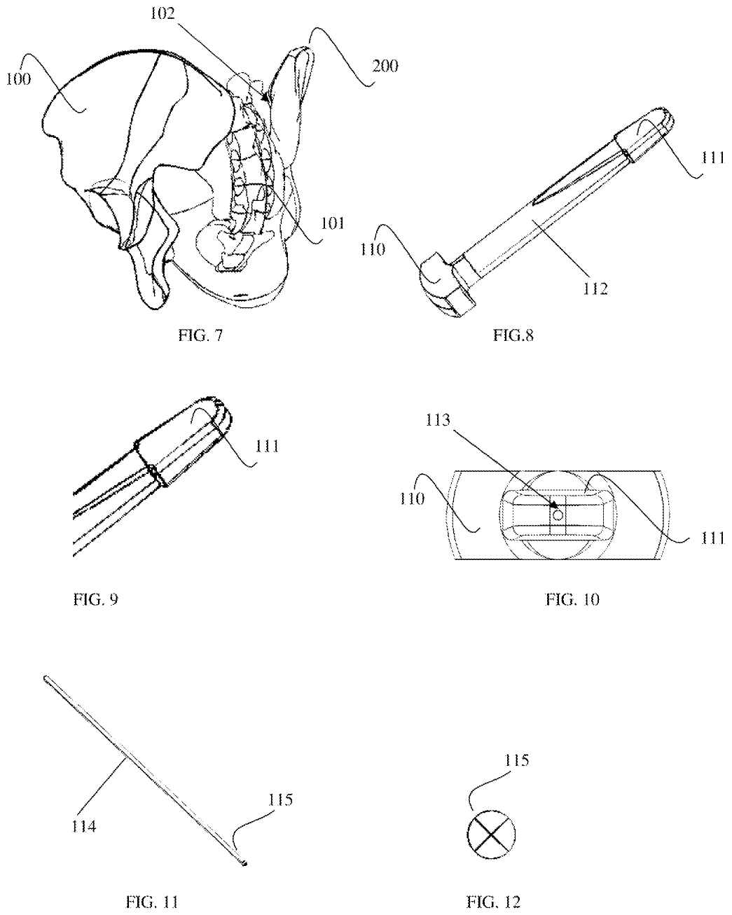

The implements may include a joint probe capable of being used to locate an insertion point in a targeted joint for a fusion implant. The joint probe may have a hollow channel therethrough for inserting a guide wire into the SI joint once the joint probe is properly positioned in the insertion point. Referring to FIGS. 8-11, the joint probe may have a rounded tip 111 for locating the insertion point, a shaft 112, and a handle 110. A hollow channel 113 run through the length of the joint probe to allow a guide wire to be inserted therethrough and into the targeted joint.

The set of tools may also include guide pins for securing the exposure device to the SI joint and the ilium. Referring to FIGS. 11-12, the guide pin 115 may have shaft that corresponds to the central channel of the joint probe and may be inserted into the targeted joint through the dilator, to guide tools and implements subsequently positioned in the targeted joint.

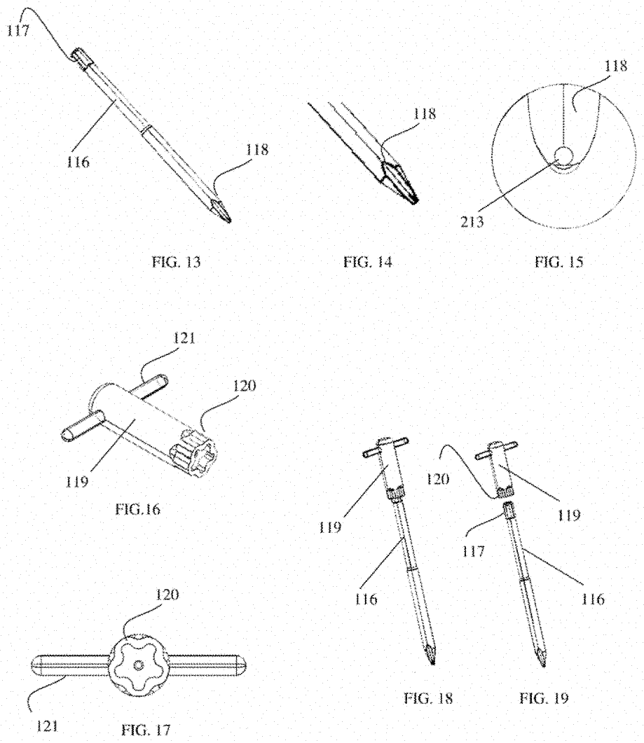

The set of surgical implements may include one or more dilators, which may be any device or structure capable of dilating an incision made in a human or other animal. FIGS. 13-19 illustrate an example of a dilator system that may be included in the present invention. Dilator 116 may be made of any suitable material and may have any suitable dimensions and configuration. In the depicted example, dilator 116 has a distal end 117, a proximal end 118, and a shaft therebetween. Proximal end 118 may have any configuration suitable to dilate an opening or incision, for example an incision made by a pin or wire in the patient's flesh and dilate that incision to increase its size. The proximal end 118 may be tapered, coming to a point at its end. The distal end 117 may be faceted, allowing it to be engaged with a grooved end 120 of a T-handle 119. A dilator assembly, an example of which is shown in FIGS. 18 and 19, allows the dilator to be spun or otherwise manipulated to adjust the size of an incision. The dilator 116 (and the t-handle 119) may have a central channel 213 running down its length that may allow a guide wire or pin to be inserted therethrough into the incision.

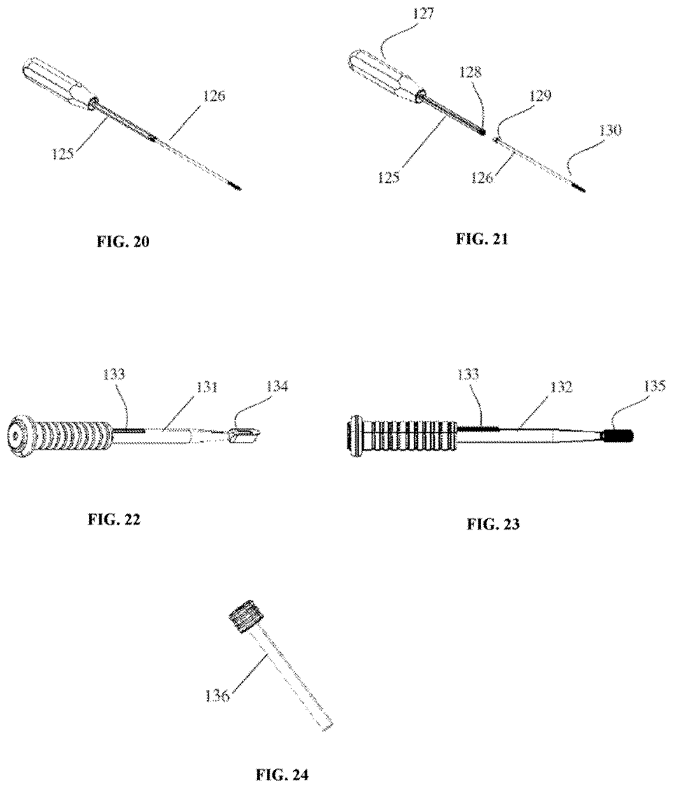

Fixing pins 126 and a fixing pin handle 125, as shown in FIGS. 20-21, may be included in the set of instruments that correspond to the pin guide slots in other instruments. For example, in some embodiments, the cannula may include guide slots through which the pins may be inserted. The guide pins may also have a sharp and/or threaded end 115 for piercing bone and other tissues. The fixing pins can be used to secure the cannula or other instruments in a desired position over the targeted joint or the ilium.

A chisel 131, as shown in FIG. 22, may be included in the set to provide an opening in the bone with desired dimensions on its distal tip 134 to better facilitate entry for the joint fusion implant. The distal tip 134 may have a tapered nose in order to self-distract its way in between the articular surfaces of bones in the targeted joint (e.g., the sacrum and ilium). The distal tip 134 may also have cutting edges to dig into the bone and remove it from the surgical site. The chisel may be used or used to aid in penetrating the cortical tissue of the sacrum and ilium to allow anchoring portions of the joint fusion implants of the present invention to penetrate the bone tissue and thereby anchor the joint fusion implant in the joint. The distal tip 134 may also have a containment device for removal of surgical site bone and windows may exist in the containment device to collect bone debris and remove the bone debris after removal from surgical site. The distal tip 134 may be undersized to a joint fusion implant to be inserted in order to ensure full bony contact on all sides of the fusion site. The chisel may have an outer diameter, as seen in FIG. 22 that matches the inner diameter of a cannula 250 to keep the chisel directed in an axial plane for desired implant preparation. The chisel may have a timing feature 133 operable to mate with a female timing feature on the inside of the cannula 250 or other incision exposure instrument to keep the chisel from plunging too far into the surgical site and to further keep the chisel in the proper orientation for desired implant site preparation.

Additionally, a rasp 132, as shown in FIG. 23, may be included in the set to provide an opening in the bone with desired dimensions on its distal tip 135 to better prepare the bone for a fusion site for the joint fusion implant. The distal tip 135 may have a tapered nose in order to self-distract its way in between the articular surfaces of bones in the targeted joint (e.g., sacrum and ilium). The distal tip 135 may have aggressive teeth which can scrape the bone to help prepare the bone surface for receiving anchoring portions of joint fusion implants of the present invention. The rasp may have an outer diameter as seen in FIG. 23, that matches the inner diameter of the cannula 250 or other incision exposure instrument to keep the rasp directed in an axial plane for desired implant preparation. The rasp may have a timing feature 133 that mates with a female timing feature on the inside of the cannula 250 or other incision exposure instrument to keep the instrument from traveling too far into the surgical site and to further keep the instrument in the proper orientation for desired implant site preparation.

One or more impactors, such as impactor 136 shown in FIG. 24, may be included in the surgical implements, as well. The impactor may have a cylindrical proximal end, like a hammer. The impactor may be included in the set to facilitate secondary impaction and movement of other surgical instruments, and, optionally, to advance bone-fusion promoting materials in the targeted joint in front of and/or behind the implant to better prepare the surgical site and to promote fusion of the targeted joint. The impactor may have an outer diameter, as seen in FIG. 22, that matches the inner diameter of the cannula 250 or other incision exposure instrument to keep the instrument directed in an axial plane for desired surgical site preparation.

The surgical kits of the present invention may also include one or more of the joint fusion implants disclosed herein, and a particular kit may include an exposure device having an internal cross-section that corresponds to a shape of the one or more joint fusion implants that are included in the kit. It is to be appreciated that additional surgical tools or implements may be used with the present working channels, and that the invention is not limited to use of the implements described in this section.

### Fusion Implants

The present invention also relates to fusion implants that include fixation element(s) that mechanically secure the targeted joint (e.g., the sacroiliac joint). Additionally, the fusion implants of the present invention may compress the targeted joint and/or may deliver bone-growth promoting material into the targeted joint to facilitate the formation of a contiguous piece of bone between the bones of the targeted joint (e.g., from the sacrum to the ilium). The joint fusion implants of the present invention may be inserted and implanted into the targeted joint. The novel posterior exposure device kit and the combined fixation and stability, and fusion functionalities of the joint fusion implants of the present invention may allow for posterior approach that may eliminate the need for patient repositioning or further incisions, resulting in less surgery time, less morbidity, and improved recovery time for the patient. Thus, the surgical methods and fusion implants of the present invention allow for a minimally invasive methodology.

To create fixation and stability from a posterior implant in the targeted joint, the joint fusion implants of the present invention connect and hold the articular surfaces of two adjacent bones of a targeted joint (e.g., the sacrum and ilium) together. The fusion implants of the present invention may be used to draw the articular surfaces of the targeted join together. Additionally, the fusion implants of the present invention may include a gap, space, or cavity for holding and delivery bone growth stimulating material to stimulate fusion of the articular surfaces, such as autologous bone, allograft, BMP, etc. In some embodiments, a combination of joint compression and bone-growth materials may be combined to provide for stable and robust bone fusion. The joint fusion device may create stress and pressure on the bone tissue by mechanically drawing the bones together, thereby bridging the two articulating bones for a fusion site. This mechanism promotes bone remodeling (e.g., as according to Wolff's Law) and the bone-growth material may further aid in the bone fusion process.

In some embodiments of the fusion implant, and without limitation, the joint fusion implant may have one or more helical anchors for insertion into bone tissue. For example, and without limitation, a joint fusion implant may include a single helix or multiple helices (e.g., 2 or 3 helices) that may be concentric and/or interwoven. In some embodiments, and without limitation, the helical path of the helical fixation anchors may have a uniform diameter from their proximal end to the distal end thereof. However, in other embodiments, and without limitation, the helical path of the helical fixation anchors may taper outward from the proximal end to the distal end of the helical path to create a conical shape to the helical path. After being inserted into the targeted joint the helical implant may be rotated in a range of about 90° to about 360° (e.g. about 180° to about 270°, or any value or range of values therein) such that helical anchors may engage the articular surfaces of adjacent bones in the targeted joint (e.g., the ilium and the sacrum) so that the fusion implant can hold the bones together and stabilize the targeted joint.

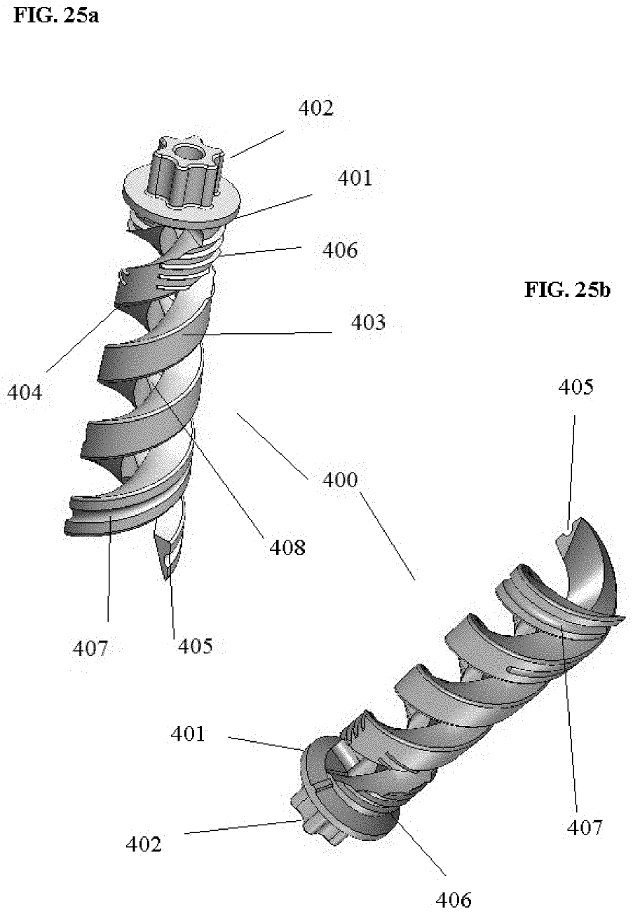

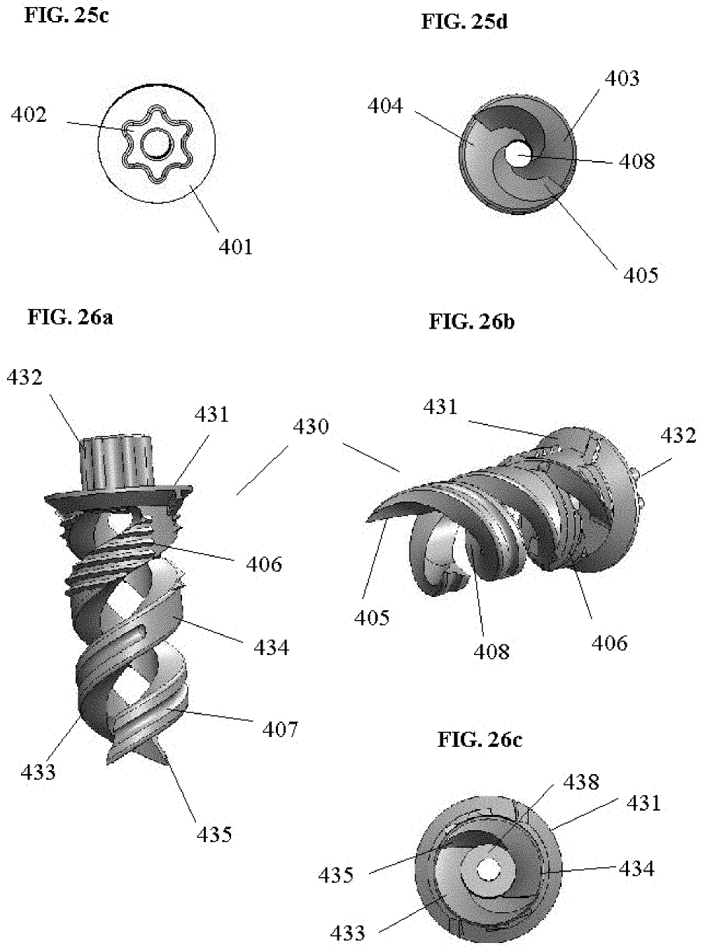

Without limiting the invention, FIGS. 25a-25d show an exemplary fusion implant 400 having two helical anchors. The implant 400 has a body 401 attached to two helical anchors 403 and 404, which may be concentric and interwoven. Each of the helical anchors 403 and 404 may have a distal cutting edge 405 that may be operable to penetrate bone tissue in joint targeted for fusion. The distal cutting edges of each of the helical anchors 403 and 404 may be on opposite sides of the fusion implant 400 such that as the implant is advanced into the targeted joint, the helical anchor 403 engages a first articulating bone (e.g., the ilium) and helical anchor 404 engages a second articulating bone (e.g., the sacrum). The body 401 of the fusion implant 400 may have a head 402 having a structure that is complementary to a bit recess in the implant inserter 340. In other embodiments, the head of the joint fusion implant may include notches or slots that may be engaged by an implant inserter having grasping mechanisms complementary thereto. The implant 400 may have an outer diameter in a range of about 8 mm to about 20 mm (e.g., about 12 mm to about 18 mm, or any other value or range of values therein).

The joint fusion implant 400 may have a central canal 408 operable to hold bone growth-promoting materials that provides for mechanical stability and promotes the formation of a contiguous piece of bone between two bones in the targeted joint (e.g., the sacrum and the ilium).

Each of the anchors 403 and 404 may have outwardly facing secondary fixation elements to improve the bite and purchase of the joint fusion implant 400 when it is implanted into the joint. For example, the anchors 403 and 404 may having external threading on at least a portion of the outer surface of the anchors 403 and 404. The external threading 406 provides further fixation structures that improve the purchase and reliability of the position of the fusion in the bone tissue.

The helical anchors 403 and 404 may also include further secondary fixation elements to assisting in the engagement of the anchors 403 and 404 with the bone. For example, the anchors 403 and 404 may further include outwardly facing fluting along the length of at least a portion of the outer surface of the anchors 403 and 404. The fluting 407 may both (1) allow bone tissue displaced by the implantation of the joint fusion implant to be passed along the fluting provide a pathway for expulsion from the surgically formed void in the targeted joint, and (2) improve the bite and purchase of the joint fusion implant 400 when it is implanted into the targeted joint.

The anchors 403 and 404 may also have a distally increasing radius. Each of the anchors 403 and 404 may follow a concentric, but expanding spiral pattern from their respective proximal ends attached to the body 401 to their respective distal ends that terminate at distal cutting edges 405, with each turn of the spiral pattern having a larger radius than the preceding turn. As a result, the diameter of the interwoven anchors 403 and 404 increases incrementally from the body 401 to the distal cutting edges 405. The retrograde tapering of the spiral pattern of the anchors 403 and 404 provides a mechanism for drawing together the articulating surfaces of the articulating bones of the targeted joint as the joint fusion implant is driven into the targeted joint. As the implant inserter 340 rotates the joint fusion implant 400 into the targeted joint, the anchors dig into the prepared bone tissue, drawing the articulating surfaces together do to the retrograde angling of the anchors 403 and 404.

In other embodiments, and without limitation, the joint fusion implant may have anchors whose spiral pattern has a consistent diameter throughout the length of the joint fusion implant. FIGS. 26a-26d show an exemplary fusion implant 430 having two helical anchors. The implant 430 has a body 431 attached to two helical anchors 433 and 434, which may be concentric and interwoven. Like the embodiment shown in FIGS. 25a-25d, each of the helical anchors 433 and 434 may have a distal cutting edge 435 that may be operable to penetrate bone tissue in joint targeted for fusion. The distal cutting edges of each of the helical anchors 433 and 434 may be on opposite sides of the fusion implant 430 such that as the implant is advanced into the targeted joint, the helical anchor 433 engages a first articulating bone (e.g., the ilium) and helical anchor 434 engages a second articulating bone (e.g., the sacrum). The body 431 of the fusion implant 430 may have a head 402 having a structure that is complementary to a bit recess in the implant inserter 340. In other embodiments, the head of the joint fusion implant may include notches or slots that may be engaged by an implant inserter having grasping mechanisms complementary thereto. The implant 430 may have an outer diameter in a range of about 8 mm to about 20 mm (e.g., about 12 mm to about 18 mm, or any other value or range of values therein). The joint fusion implant 430 may have a central canal 438 operable to hold bone growth-promoting materials that provides for mechanical stability and promotes the formation of a contiguous piece of bone between two bones in the targeted joint (e.g., the sacrum and the ilium).

Each of the anchors 433 and 434 may have outwardly facing secondary fixation elements. For example, the anchors 433 and 434 may include threading on at least a portion of the outer surface of the anchors 433 and 434 to improve the bite and purchase of the joint fusion implant 430 when it is implanted into the joint. The helical anchors 433 and 434 may also include further secondary fixation elements. For example, the helical anchors 433 and 434 may include outwardly facing fluting along the length of at least a portion of the outer surface of the anchors 403 and 404.

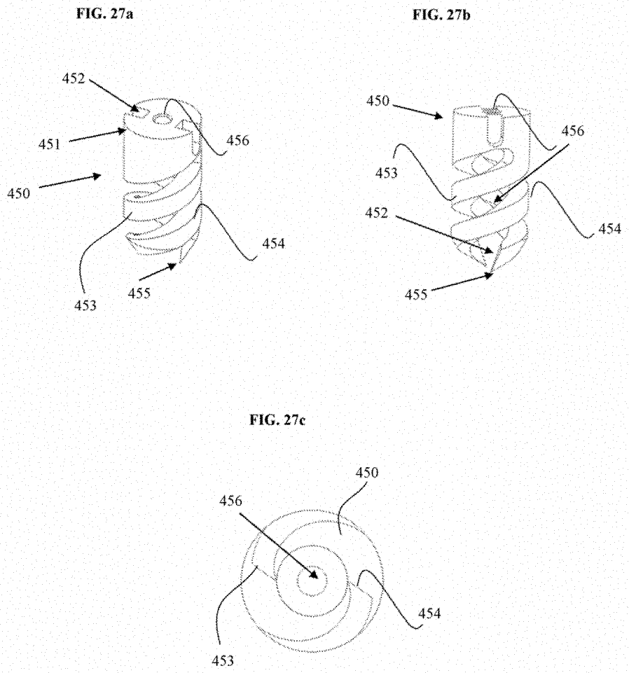

In other embodiments, and without limitation, the joint fusion implant may have one or more helical anchors that have a smooth exterior, without threading or fluting, as show in FIGS. 27a-27c. For example, and without limitation, the joint fusion implant 450 may include two helical anchors 453a and 453b having a polygonal cross-sections (may be round or elliptical in other embodiments) that concentrically spiral around one another. The fusion implant 450 has a body 451 attached to the helical anchors 453 and 454 at their proximal ends. The helical anchors 453 and 454 may each have a distal cutting edge 455 that may be operable to penetrate bone tissue in joint targeted for fusion. The body 451 of the fusion implant 450 may have notches or slots 452 in a perimeter thereof that may be engaged by a fusion implant inserter or driver as described herein (e.g., by an inserter having fork arms). Alternatively, the joint fusion device may have a head having a different engagement structure, such as a star-shaped driver pattern.

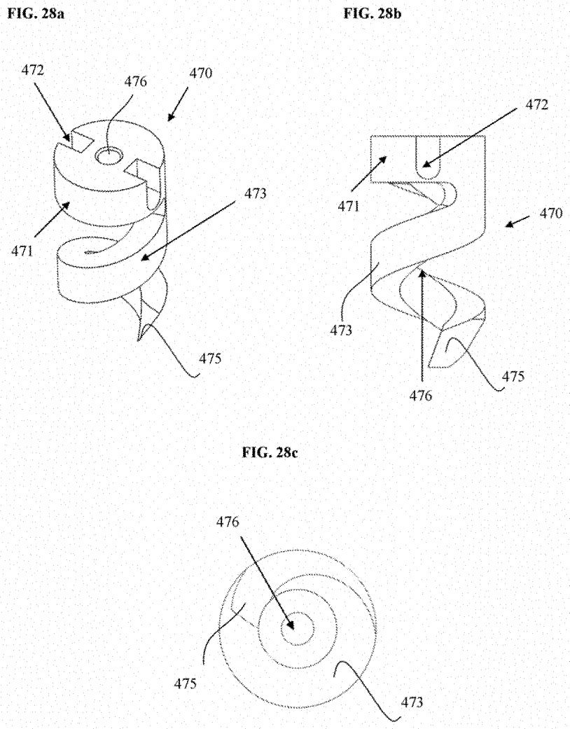

In still other embodiments, and without limitation, the fusion implant may have a single helix attached to the body of the fusion implant. For example, and without limitation, FIGS. 28a-28c illustrate an exemplary joint fusion implant 470 that may include a single helical anchor 473. The fusion implant 470 has a body 471 attached to the helical anchor 473, which may have a distal cutting edge 475 that may be operable to penetrate bone tissue in joint targeted for fusion. The body 471 of the fusion implant 470 may have notches or slots 472 in a perimeter thereof that may be engaged by a fusion implant inserter or driver as described herein (e.g., by an inserter having fork arms). Alternatively, the joint fusion device may have a head having a different engagement structure, such as a star-shaped driver pattern.

### Surgical Methods

In some embodiments, the methods of the present invention substantially fuse the targeted joint (e.g., the SI joint), such that movement in the joint is minimized or substantially eliminated, thereby diminishing or substantially eliminating the patient's pain and discomfort. More specifically, an improved, combined approach for both mechanical holding and surgical fusion using the novel surgical tools and tool sets of the present invention, as described above. Specifically, with respect to some embodiments, an approach is described to address the targeted joint (e.g., an SI joint) through a posterior approach. In some embodiments, and without limitation, the surgical fusion of the sacrum and an ilium may be accomplished with a posteriorly inserted fusion implant device alone. In other embodiments, and without limitation, surgical fusion may be accomplished with the delivery of a plurality of fusion implant devices into the SI joint. The fusion may be delivered to the SI joint, placed between the sacrum and ilium and positioned about between a posterior inferior iliac spine and the sacrum of the sacroiliac joint. In some embodiments, a second fusion implant may be delivered into the sacroiliac joint at a position about between a posterior superior iliac spine and a superior lateral aspect of the sacrum of the sacroiliac joint.

In some embodiments, and without limitation, the method may involve the posterior insertion of a fusion implant, including the steps of creating an incision proximal to the patient's SI joint, introducing a guide wire, dilating the incision, inserting a cannula into the incision over the dilator and guide wire, creating a void in the SI joint, inserting a washer device, and inserting a fusion implant into the void through the washer device such that it engages with the articular surfaces of the sacrum and ilium.

Some embodiments, without limitation, include some or all of the following steps, preparing the patient for surgery (e.g., positioning the patient in a prone position to provide the surgeon access to the SI joint, general or local anesthesia, and the like), locating the SI joint and an incision point for access to the SI joint (e.g., by blunt finger palpation), insertion of a pin or wire to create an incision, insertion of a dilator over the pin and impacting the dilator to dilate the incision to a width through which instruments may be passed, inserting a cannula over the dilator, securing the cannula in position (e.g., with fixing pins), removing the dilator, inserting a drill bit apparatus through the working channel, using the drill bit apparatus in the working channel to displace bone in the SI joint thereby creating a void, removing the drill bit apparatus, pairing a washer device with a washer driver, inserting the washer device into the sacroiliac joint utilizing the washer driver, removing the washer driver from the washer once the washer is positioned at the sacroiliac joint, loading a fusion implant onto an inserter and inserting the fusion implant and inserter through the cannula and through a ring of the washer device until the implant is positioned proximal to the void in the patient's SI joint, driving the fusion implant into the joint to engage the articular surfaces of the SI joint (e.g., by rotating and driving the fusion implant forward into the joint), breaking away guidance tabs on the washer device, removing all instruments, and closing the incision.

Embodiments of the methods of the present invention (and tools of the present invention) are now further described with reference to the Figures. Although the methods are described with respect to the use of certain tools, the methods of the present invention may be performed in addition to or in conjunction with one or more of the known other tools used in the relevant art.

FIGS. 34a-34o illustrate a surgical procedure for fusing an SI joint with one or more fusion implants inserted in the SI joint. The procedure includes positioning a patient in the prone position and administering either a local or general anesthetic. Blunt finger palpation may be used to locate the patient's iliac wing and the SI joint. As shown in FIGS. 1-7, the SI joint 102 is located between the iliac wing 100 and the sacrum 101 at the base of the pelvis. The SI joint is fully enclosed between the iliac wing 100 and the sacrum 101 and occluded for direct visualization by the iliac wing 100. Additionally, the iliac crest 200, the posterior superior and inferior iliac spines, and the pedicle 104 of vertebrae 51 can be observed in this view. The iliac crest 200 may provide a posterior landmark for the entry point of the exposure device of the present invention at the posterior iliac crest, and can be palpated to find the general location of the SI joint. Alternatively, suitable locations for an incision may be determined by imaging methods (e.g., x-ray), or any other suitable method.

FIGS. 8-24 show tools that may be used to establish an incision and assist in establishing a void in the sacroiliac joint. A joint probe 112 may be used to identify the insertion area on the posterior side of the SI joint. The area of the SI joint may be probed until the rounded geometry of the joint probe 112 finds or drops into the proper position in the SI joint, where an incision may be properly made. Subsequently, a guide pin 114 may be inserted through a central channel in the joint probe 112 and into the patient to create an incision in the SI joint. Alternatively, the incision may be made by any suitable method, including scalpel or other cutting or dissection tool. The incision may be made proximal to the patient's SI joint, allowing the joint to be accessed by the exposure device. The guide pin 114 may be advanced until its proximal end is in contact with the SI joint or at least partially within SI joint.

A dilator may be used to dilate the incision. As an example, dilator 116 may be slotted over guide pin 114 through a central channel running the length of the dilator 116. The proximal end of the dilator 116 may be slotted over the guide pin 114, and dilator 116 may then be advanced to or near the SI joint through the incision. As dilator 116 enters the incision, the tapered end 118 pushes the patient's flesh and tissue aside, thereby dilating incision to accommodate an exposure device as described herein. A joint cutting assembly that includes the dilator 116 and a T-handle 120 engaged with a distal end of the dilator 116 may be used to further drive the dilator 116 into the incision to a desired depth to sufficiently expose the SI joint. Alternatively, an impactor (not shown) may be used to further drive the dilator 116 into the incision to a desired depth.

FIG. 34a illustrates the placement of the cannula 250 over the guide wire 114. The cannula 250 can be positioned over the coupled dilator 116 (not shown in FIG. 34a) and the guidewire 114 in the incision and the dilator 116 may be removed from the incision. The cannula is advanced toward SI joint through incision until proximal end 251 is in contact with the SI joint or proximal to the SI joint and in contact with the sacrum and/or ilium. The cannula 250 has distal end 251 with a round geometry and/or a tapered rounded profile that is operable to distract the SI joint with minimal damage to soft and connective tissue in and around the posterior side of the SI joint. The cannula 250 may also have an arched cut-away portion 251a that accommodates the bony anatomy of the SI joint (e.g., the iliac crest). It is to be appreciated that the cannula may have other distal geometries, e.g., where there is no cut-away and the distal end has a complete circular circumference. It should be understood that the cannula may have other cross-sectional shapes as well (e.g., elliptical, polygonal [pentagonal, hexagonal, etc.], Reuleaux shapes, and other applicable shapes), e.g., to accommodate the shape of various fusion implants. The cannula may also further dilate incision.

The cannula 250 may include exterior features (not shown) that allow for the stabilization of the cannula in the SI joint. For example, the cannula may include holes, slots or tabs through which fixing pins 126 may be slotted to stabilize the cannula 250. In some embodiments, and without limitation, fixing pins 126 can penetrate the skin and/or flesh and tissue of a human. It is to be appreciated that any suitable method of stabilizing exposure device may be used. Dilator 116 may be removed from the cannula 250 either before or after the fixing pins 126 are inserted. In other embodiments, and without limitation, the exposure device may be stabilized by attachment to a surgical or stabilizing arm to hold the exposure device in a static and stable position.

FIGS. 34c-34d illustrate insertion of drill bit 260 into the incision through the cannula 250. The drill bit 260 may be connected to a power drill configured for medical procedures. The drill bit apparatus 260 may have cylindrical outer walls that allow the drill bit apparatus 260 to freely spin within the hollow barrel of the cannula 250. The cylindrical outer wall may comprise a low-friction material that facilitates smooth spinning of the drill within the hollow barrel. The proximal end of the drill bit apparatus 260 may be inserted into the cannula 250 over guidewire 114 and may be advanced to a predetermined point. In some examples, and without limitation, the distal end of drill bit apparatus 260 extend past the distal end 251 of the cannula 250 by a pre-determined amount that is equal to or less than a length of an insertable portion of a fusion implant (e.g., fusion implant 400, 450, 475, etc.). The drill bit 260 may have a shoulder 266 that provides indication of the depth to which the drill should be advanced into the SI joint. When the shoulder 266 meets the proximal end of the cannula 250, the drill bit 260 may be advanced to the desired pre-determined depth. When advanced into the SI joint, the drill bit 260 may create a void in the patient's SI joint by displacing portions of sacrum and ilium. In such examples, the drill bit 260 may be configured such that it will contact the patient's SI joint at a desired portion of the joint and, once activated, will create a void of a desired depth. The void may be configured to receive a fusion implant as described herein or other joint repairing appliance or bone graft for fusing the SI joint. Other joint repairing appliances or apparatus may include a polyether ether ketone (PEEK) implant, a titanium implant, etc. As an example and without limiting the invention, the implant may be a fusion implant like one of those shown in FIGS. 25a-28b.

The present method may utilize several implements that may be inserted through the cannula 250 and into the void in the SI joint to prepare the void for receiving a fusion implant. For instance, a box chisel 131 and/or a rasp 132 may be inserted into the void through the cannula 250 to expand and clear tissue from the void to facilitate a clean and efficient insertion of the fusion implant into the void (see, e.g., FIGS. 8-24).

As shown in FIGS. 34e-34f, a washer device 310 may be paired with a washer driver 330 to prepare the washer for insertion into the SI joint. The washer device 310 includes a washer ring 311 at its distal end that may include lateral tangs 312a and 312b for penetrating tissues in the SI joint and establishing a stable purchase of the washer device 310 before a joint fusion implant is inserted and implanted into the SI joint. The washer device 310 may further include guidance tabs 315a and 315b that connect to the washer ring 311 at break-away connection points 314a and 314b. The guidance tabs 315a and 315b may be operable to be engaged with a washer driver 330 in order to couple the washer device 310 and the washer driver 330 together. The guidance tab may include coupling channels 316a and 316b (see, e.g., FIGS. 31a and 31c) that may be engaged with coupling pegs 331a and 331b of the washer driver 330. As shown in FIG. 34e-34f, the washer driver 330 may be nested within the central channel of the washer device 310 and the coupling pegs 331a and 331b may be slotted into the coupling channels 316a and 316b to secure the washer device 310 and washer driver together for insertion of the washer 310 into the SI joint through the cannula 250.

FIGS. 34g-34h show the process of insertion of the coupled washer device 310 and washer driver 330 being inserted into the cannula 250 over the guidewire 114 and then driven through the cannula 250 (e.g., by mallet or other driving tool) such that the washer is inserted into the SI joint. The lateral tangs 312a and 312b are operable to penetrate tissue in the targeted joint by being driven into the joint, e.g., by impaction. The lateral tangs may include biting or gripping features (e.g., serrations, barbs, etc.) that provide for structures that facilitate firm lodging in the tissue (e.g., connective tissues, cartilage, prepared bone surface, etc.). Such biting or gripping features may have a retrograde angle directed toward the proximal end of the washer 310 to prevent dislodgement of the washer 311 from the targeted joint. The lateral tangs 312a and 312b may be arranged such that they are diametrically or substantially diametrically positioned on washer ring 311 such that they can be driven between two adjacent articular surfaces of bones in the target joint, allowing penetration of the joint by both of the tangs 312a and 312b. The washer establishes a stable purchase in the SI joint. The ring 311 of the washer may have a receiving surface on its proximal side for engaging with the body of the fusion implant. The distal surface of the fusion implant may have a complementary surface to the proximal surface of the ring structure 311, such that the body of the fusion implant nests snugly and evenly in the ring structure 211.

FIGS. 34i-34j illustrate the use of a fusion implant inserter 340 to insert a fusion implant (e.g., fusion implant 400) into the void in the SI joint. The inserter 340 may be inserted into the cannula 250 once the washer driver 330 has been decoupled from the guidance tabs 315a and 315b of the washer device 310 and removed from the cannula 250. Inserter 340 is operable to engage with a joint fusion implant and insert and implant the joint fusion implant into the targeted joint. The implant inserter 340 may be engaged with the proximal end of a joint fusion implant and then used to advance the joint fusion implant through the cannula 250 and into the targeted joint. The implant inserter 340 may have a distal end 341 configured to attach to the joint fusion device, e.g., having a bit-receiving recess (e.g., star, hex, or other functional shape) for receiving a complementary head of a joint fusion device. The washer inserter 340 may have a mechanism for tightening the engagement of the bit recess with the head of the joint fusion implant. For example, an actuator operable by the medical personnel may control a spring-operated element, e.g., in the bit recess that reversibly applies pressure to the head of the joint fusion implant to maintain the joint head in the bit recess.

The implant inserter 340 may have a recessed portion 342 along its proximal shaft having a cutaway 343 that allows the medical personnel to monitor the depth to which the implant inserter 340 penetrates the targeted joint (e.g., the distal end of the cutaway can be used as a marker). The shoulder 343 may act as a marker to be used in conjunction with the markers on the guidance tabs 315a and 315b to determine when the implant inserter 340 has reached the desired depth to deliver a joint fusion implant into the targeted joint. For example, at least one washer tab may be marked to indicate the point to which the distal end of the cutaway 343 of the implant inserter should be advanced to completely insert he fusion implant into the targeted joint. In other examples, at least one washer tab may include two marks, a proximal mark to indicate the point to which the distal end of the cutaway 343 should be advance to place the distal end of the fusion implant at superficial aspect of the bones in the joint, and a distal mark to indicate the point to which the shoulder 343 of the implant inserter should be advanced to completely insert the fusion implant into the targeted joint. A first proximal position marker on a guidance tab 312a of the washer device 310 may indicate to the medical personnel that the joint fusion device is at the superficial aspect of the targeted joint when the shoulder 343 of the implant inserter 340 is aligned therewith.

Once the inserter 340 has been advanced to the predetermined position at which the distal end of the joint fusion implant is at the superficial aspect of the SI joint, linear advancement of the inserter 340 is arrested, and the fusion implant 400 is positioned to be rotated while it is advanced, such that it engages and penetrates the articular surfaces of the bone to fix the articular surfaces relative to one another. The medical personnel may then rotate the implant inserter 340 using a driving tool to cause the fixation element(s) of the joint fusion implant to penetrate the bone tissue of the targeted joint. The implant inserter 340 may have a Jacob's chuck connection 435 so that it may be attached to a driving tool. The medical personnel may continue rotating the implant inserter 340 until the distal end of the cutaway 343 (or other marker) is aligned with a second distal position marker on a guidance tab 312a of the washer device 310, which indicates that the joint fusion implant is fully implanted into the bone tissue.

When the fusion implant is fully advanced, the distal portion of the body of the implant may be nested in the complementary surface of the ring 311 of the of the washer device 310. This provides a stable, even and snug engagement and position between the washer device 310 and the fusion implant.

Once the fusion implant has been fulling inserted into the SI joint, the inserter 340 may be disengaged from the fusion implant and removed from the cannula 250, and the cannula 250 and guidewire 114 can be removed from the SI joint, leaving the washer device 310 exposed in the incision. FIGS. 34k and 34l show the process of removing the guidance tabs 315a and 315b from the washer ring 311, which is to remain engaged with and implanted in the SI joint. The guidance tabs 315a and 315b may be operable to be removed from the washer ring 311 at connection points 314a and 314b. For example, the material of the guidance tabs 315a and 315b may be sufficiently thin and/or pre-treated (e.g., scored, thermally treated, chemically treated, or otherwise prepared) such that the guidance tabs may be snapped or broken off at the connection points 314a and 314b such that they can be removed from the washer ring 311 and the incision over the SI joint. In other examples, the guidance tabs 315a and 315b can be removed by other techniques, e.g., by a shearing or cutting tool.

FIGS. 34k and 341 illustrate an example of how the guidance tabs 315a and 315b may be removed. The ring of material joining the proximal ends of the guidance tabs 315a and 315b may be cut or otherwise sheared using a cutting tool. Subsequently, the guidance tabs may be rotated outward from the washer ring along a plane perpendicular to the washer ring 311, such that shearing force is applied at the connection points 314a and 314b causing the guidance tabs 315a and 315b to break away from the washer ring 311 at connection points 314a and 314b. The guidance tabs may then be removed from the incision, and the tissues in the incisions may then be sutured, to facilitate healing.

FIG. 34m provides a view of a fusion implant in position in the SI joint after surgical implantation. The pelvis is presented as semi-transparent for illustration purposes. The fusion implant is positioned about between a superior lateral aspect of the sacral wing and the posterior superior iliac spine of the ilium. This is an appropriate and effective location for implantation between the articular surfaces of the SI joint. However, the present invention is not limited to such placement in the SI joint. For example, the fusion implant may be positioned at or near an inferior aspect of the sacroiliac joint.

Additionally, the present invention is not limited to methods that insert a single fusion implant into an SI joint. FIG. 34n shows the placement of two fusion implants in an SI joint, a first implant positioned about between a superior lateral aspect of the sacral wing and the posterior superior iliac spine of the ilium, and a second implant positioned at or near an inferior aspect of the sacroiliac joint.

It is also to be appreciated that the tools, devices, and methods disclosed herein are not limited to SI joint fusion procedures, and may have other beneficial applications. It is to be further appreciated that the tools, devices, and methods disclosed herein can be utilized in SI joint fusion procedures on both SI joints of a patient simultaneously.

It is to be understood that variations and modifications of the present invention may be made without departing from the scope thereof. It is also to be understood that the present invention is not to be limited by the specific embodiments disclosed herein, but only in accordance with the appended claims when read in light of the foregoing specification.

What is claimed:

1. A medical instrument kit, comprising:
a. a joint fusion implant having a central body and at least one implant fixation element for engagement with bone tissue in at least one articular surface of a targeted joint; and
b. a washer device having a washer ring having at least one washer fixation element for engagement with said targeted joint.

2. The kit of claim 1, wherein said washer device includes at least one distal tang that is operable to be inserted into the targeted joint and is operable to stabilize medical instrumentation for inserting said joint fusion implant.

3. The kit of claim 1, further comprising guidance tabs attached to said washer device for guiding the insertion of said joint fusion implant into said targeted joint.

4. The kit of claim 3, wherein said guidance tabs are fixedly attached to said washer with break-away connections that allow the guidance tabs to be removed from the washer device once said washer device is engaged with said targeted joint.

5. The kit of claim 1, further comprising a washer driver operable to be coupled with said washer device prior to the insertion of said washer device into said targeted joint and operable to be removed from said washer device after said washer device is inserted into said targeted joint.

6. The kit of claim 1, further comprising a cannula through which said coupled washer device and washer driver are passed to advance the coupled washer device and washer driver to said targeted joint.

7. The kit of claim 1, wherein said at least one implant fixation element is operable to engage bone tissue in articular surfaces of two bones in said joint to fix said articular surfaces in position relative to each other.

8. The kit of claim 1, wherein said at least one implant fixation element includes at least one secondary fixation element thereon.

9. The kit of claim 8, wherein said at least one secondary fixation element includes a protrusion on an outer edge of said at least one implant fixation element operable to engage with an articular surface of said targeted joint.

10. The kit of claim 9, wherein said at least one secondary fixation element includes threading on an outer edge of said at least one implant fixation element.

11. The kit of claim 8, wherein said at least one secondary fixation element includes fluting on an outer edge of said at least one implant fixation element.

12. The kit of claim 1, wherein said at least one fixation element comprises a helical anchor having a sharp end for piercing said bone tissue in an articular surface of at least one of said sacrum and said ilium.

13. The kit of claim 1, wherein said fusion implant comprises a body having two helical anchors for engaging with said bone tissue, and a cavity is formed between said two helical anchors.

14. The kit of claim 13, wherein said two helical anchors are arranged in an interwoven double helix pattern, and each helical anchor has a sharp end for piercing said bone tissue.

15. The kit of claim 1, wherein said joint fusion implant comprises a cavity for holding a bone fusion-promoting material.

16. The kit of claim 6, wherein said incision is over a sacroiliac joint of a human patient and said surgical tool is for insertion into said sacroiliac joint, and said joint fusion implant is operable to be inserted through said cannula and said washer ring and between the articular surfaces of the sacrum and ilium in said sacroiliac joint.

17. The kit of claim 3, further comprising:
a. a drill operable to be passed through said cannula into said incision and drilling a void through the connective tissues in said targeted joint; and
b. an inserter having a proximal end configured to attach to said joint fusion implant, said inserter being operable to pass said fusion implant through said washer ring and into said targeted joint.

18. The kit of claim 17, wherein the drill comprises a drill bit having an outer diameter that is substantially equal to an outer diameter of said washer, allowing the washer to be inserted in shallow portion of targeted joint.

19. The kit of claim 1, wherein the washer has an outer diameter that is greater than the distance between the articular surfaces of the targeted joint, such that the washer may be stably positioned superficially to the targeted joint.

20. A medical device for treating a targeted joint, comprising:
a. a washer device having a washer ring having at least one washer fixation element for engagement with said targeted joint; and
b. guidance tabs attached to said washer device for guiding the insertion of said joint fusion implant into said targeted joint.

21. The device of claim 20, wherein said washer device includes at least one distal tang that is operable to be inserted into the targeted joint and is operable to stabilize medical instrumentation for inserting said joint fusion implant.

22. The device of claim 20, wherein said guidance tabs are fixedly attached to said washer with break-away connections that allow the guidance tabs to be removed from the washer device once said washer device is engaged with said targeted joint.

23. The device of claim 20, further comprising at least one secondary fixation element on said at least one washer fixation element

24. The device of claim 23, wherein said at least one secondary fixation element includes a protrusion on an outer edge of said at least one washer fixation element operable to engage with an articular surface of said targeted joint.

25. The device of claim 24, wherein said at least one secondary fixation element includes a barb on an outer edge of said at least one washer fixation element.

26. The device of claim 20, further comprising at least one secondary fixation element on said washer device.

27. The device of claim 26, wherein said at least one secondary fixation element includes a protrusion on a ring structure of said washer device.

28. The device of claim 27, wherein said at least one secondary fixation element includes a barb on said ring structure of said washer device.

29. The device of claim 20, wherein said guidance tabs are operable to be engaged with a washer driver operable to couple said washer device and said washer driver prior to the insertion of said washer device into said targeted joint, said washer driver being operable to be removed from said washer device after said washer device is inserted into said targeted joint.

30. The device of claim 29, wherein said coupled washer device and washer driver are operable to be passed through a cannula to advance the washer device into said targeted joint.

31. The device of claim 20, wherein said washer device includes a ring structure having an outer diameter that is greater than the distance between the articular surfaces of the targeted joint, such that the washer may be stably positioned superficially to the targeted joint.

32. The device of claim 20, wherein said washer device includes a ring structure having an inner diameter that is slightly greater than the outer diameter of a fusion implant, such that the fusion implant may be passed in a co-axial path through the inner diameter of the ring structure and between articular surfaces of the targeted joint.

33. The device of claim 20, wherein the outer diameter of said washer is substantially equal to outer diameter of a drill bit for forming a void in said targeted joint, allowing the washer to be inserted in a shallow portion of targeted joint after the drill bit forms said void.

34. A medical instrument kit, comprising:
a. a joint fusion implant having a central body and at least one implant fixation element for engagement with bone tissue in at least one articular surface of a targeted joint; and
b. a washer device having a washer ring having at least one washer fixation element for engagement with said targeted joint, and guidance tabs attached to said washer device for guiding the insertion of said joint fusion implant into said targeted joint.

35. The kit of claim 34, wherein said washer device includes at least one distal tang that is operable to be inserted into the targeted joint and is operable to stabilize medical instrumentation for inserting said joint fusion implant.

36. The kit of claim 35, wherein said guidance tabs are fixedly attached to said washer with break-away connections that allow the guidance tabs to be removed from the washer device once said washer device is engaged with said targeted joint.

37. The kit of claim 34, further comprising a washer driver operable to be coupled with said washer device prior to the insertion of said washer device into said targeted joint and operable to be removed from said washer device after said washer device is inserted into said targeted joint.

38. The kit of claim 34, further comprising a cannula through which said coupled washer device and washer driver are passed to advance the coupled washer device and washer driver to said targeted joint.

39. The kit of claim 34, wherein said at least one implant fixation element is operable to engage bone tissue in articular surfaces of two bones in said joint to fix said articular surfaces in position relative to each other.

40. The kit of claim 34, wherein said at least one implant fixation element includes at least one secondary fixation element thereon.

41. The kit of claim 40, wherein said at least one secondary fixation element includes a protrusion on an outer edge of said at least one implant fixation element operable to engage with an articular surface of said targeted joint.

42. The kit of claim 40, wherein said at least one secondary fixation element includes threading on an outer edge of said at least one implant fixation element.

43. The kit of claim 40, wherein said at least one secondary fixation element includes fluting on an outer edge of said at least one implant fixation element.

44. The device of claim 34, further comprising at least one secondary fixation element on said at least one washer fixation element

45. The device of claim 44, wherein said at least one secondary fixation element includes a protrusion on an outer edge of said at least one washer fixation element operable to engage with an articular surface of said targeted joint.

46. The device of claim 44, wherein said at least one secondary fixation element includes a barb on an outer edge of said at least one washer fixation element.

47. The kit of claim 34, wherein said at least one fixation element comprises a helical anchor having a sharp end for piercing said bone tissue in an articular surface of at least one of said sacrum and said ilium.

48. The kit of claim 47, wherein said fusion implant comprises a body having two helical anchors for engaging with said bone tissue, and a cavity is formed between said two helical anchors.

49. The kit of claim 48, wherein said two helical anchors are arranged in an interwoven double helix pattern, and each helical anchor has a sharp end for piercing said bone tissue.

50. The kit of claim 34, wherein said joint fusion implant comprises a cavity for holding a bone fusion-promoting material.

51. The kit of claim 38, wherein said incision is over a sacroiliac joint of a human patient and said surgical tool is for insertion into said sacroiliac joint, and said joint fusion implant is operable to be inserted through said cannula and said washer ring and between the articular surfaces of the sacrum and ilium in said sacroiliac joint.

52. The kit of claim 34, further comprising:
a. a drill operable to be passed through said cannula into said incision and drilling a void through the connective tissues in said targeted joint; and
b. an inserter having a proximal end configured to attach to said joint fusion implant, said inserter being operable to pass said fusion implant through said washer ring and into said targeted joint.

53. The kit of claim 52, wherein the drill comprises a drill bit having an outer diameter that is substantially equal to an outer diameter of said washer, allowing the washer to be inserted in shallow portion of targeted joint.

54. The kit of claim 34, wherein the washer has an outer diameter that is greater than the distance between the articular surfaces of the targeted joint, such that the washer may be stably positioned superficially to the targeted joint.

55. A joint fusion implant capable of fixing articulating bones of a targeted joint, comprising:
a. a body; and
b. at least one implant fixation element having a sharp portion for engagement with bone tissue in at least one articular surface of said targeted joint, wherein said at least on fixation element includes secondary fixation elements thereon,
wherein said at least one implant fixation element is adapted to be inserted between an intra-articular space between articular surfaces of a joint in general longitudinal alignment with a plane between said articular surfaces of said joint.

56. The implant of claim 55, wherein said fusion implant is configured to be rotated by a driving tool to insert said at least one fixation element into said bone tissue in said articular surface.

57. The implant of claim 56, wherein a proximal end of said body includes at least one receiving surface for engagement with said driving tool, said driving tool being for rotating said body and driving said at least one fixation element into said bone tissue in said at least one of said articular surfaces in said joint.

58. The implant of claim 55, wherein said fusion implant comprises a plurality of implant fixation elements, each extending from said body.

59. The implant of claim 55, wherein said at least one implant fixation element is operable to engage bone tissue in articular surfaces of two bones in said joint to fix said articular surfaces in position relative to each other.

60. The implant of claim 55, wherein said at least one secondary fixation element includes a protrusion on an outer edge of said at least one implant fixation element operable to engage with an articular surface of said targeted joint.

61. The implant of claim 60, wherein said at least one secondary fixation element includes threading on an outer edge of said at least one implant fixation element.

62. The implant of claim 60, wherein said at least one secondary fixation element includes fluting on an outer edge of said at least one implant fixation element.

63. The implant of claim 55, wherein said at least one fixation element comprises a helical anchor having a sharp end for piercing said bone tissue in an articular surface of at least one of said sacrum and said ilium.

64. The implant of claim 63, wherein said fusion implant comprises a body having two helical anchors for engaging with said bone tissue, and a cavity is formed between said two helical anchors.

65. The implant of claim 64, wherein said two helical anchors are arranged in an interwoven double helix pattern, and each helical anchor has a sharp end for piercing said bone tissue.

66. The implant of claim 55, wherein said joint fusion implant comprises a cavity for holding a bone fusion-promoting material.

67. A method for repairing a sacroiliac joint of a patient, comprising:
a. creating a first incision in the patient's skin proximal to the patient's sacroiliac joint;
b. inserting a cannula into said first incision;
c. creating a void in said sacroiliac joint;
d. passing a washer device through said cannula for placement at the superficial aspect of said targeted joint; and
e. inserting a joint fusion implant into said void through said washer device.

68. The method of claim 67, wherein creating said void comprises inserting a drill into said cannula and drilling said void through tissues in said joint, wherein the drill comprises a drill bit having an outer diameter that is substantially equal to an outer diameter of a ring structure of said washer device, wherein the washer device is operable to be inserted in shallow portion of said void.

69. The method of claim 67, wherein said washer device comprises guidance tabs attached to said washer device for guiding the insertion of said joint fusion implant into said targeted joint.

70. The method of claim 69, wherein said guidance tabs are fixedly attached to said washer with break-away connections that allow the guidance tabs to be removed from the washer device once said washer device is engaged with said targeted joint.

71. The method of claim 70, further comprising coupling a washer driver to said guidance tabs of said washer device prior to the insertion of said washer device into said targeted joint.

72. The method of claim 71, wherein passing said washer device through said cannula includes passing said coupled washer device and washer driver into said targeted joint through a washer ring.

73. The method of claim 72, further comprising removing said washer driver from said washer device, leaving said washer device engaged with said targeted joint.

74. The method of claim 73, wherein inserting a joint fusion implant into said void comprises using an implant inserter to pass said fusion implant through said washer, wherein said inserter moves said implant through said cannula in a linear, co-axial path through the cannula until said at least one fixation element reaches said washer device, and rotating said joint fusion implant into said void such that an at least one fixation element of said fusion implant engages with at least one articular surface of said joint.

75. The method of claim 74, further comprising removing said guidance tabs from said ring structure of said washer device after said fusion implant is inserted into said void.

76. The method of claim 75, wherein said guidance tabs are fixedly attached to said washer with break-away connections that allow the guidance tabs to be removed from the washer device once said washer device is engaged with said targeted joint.

77. The method of claim 67, further comprising inserting a guide wire into said sacroiliac joint of the patient, slotting a joint cutting tool over said guide wire, and placing said cannula over said guide wire and said joint cutting tool, such that said guide wire and joint cutting tool are within a barrel of said cannula and an end of said cannula is within said sacroiliac joint.

78. The method of claim 67, further comprising removing cortical tissue from said articular surfaces of said articulating bones prior to inserting said fusion implant into said void.

79. The method of claim 67, wherein said washer device has a washer ring having at least one washer fixation element for engagement with said targeted joint.

80. The method of claim 79, wherein said washer fixation element includes at least one distal tang that is operable to be inserted into the targeted joint and is operable to stabilize medical instrumentation for inserting said joint fusion implant.

81. The method of claim 67, wherein said fusion implant has at least one fixation element for engagement with bone tissue in an articular surface of at least one of the articulating bones of said targeted joint.

82. The method of claim 81, further comprising rotating said at least one fixation element to engage said bone tissue in at least one articular surface of said targeted joint.

83. The method of claim 82, wherein said at least one implant fixation element is operable to engage bone tissue in articular surfaces of two articulating bones in said joint to fix said articular surfaces in position relative to each other.

84. The method of claim 81, wherein said at least one implant fixation element includes at least one secondary fixation element thereon.

85. The method of claim 84, wherein said at least one secondary fixation element includes a protrusion on an outer edge of said at least one implant fixation element operable to engage with an articular surface of said targeted joint.

86. The method of claim 84, wherein said at least one secondary fixation element includes threading on an outer edge of said at least one implant fixation element.

87. The method of claim 84, wherein said at least one secondary fixation element includes fluting on an outer edge of said at least one implant fixation element.

88. The method of claim 83, wherein said at least one fixation element comprises a helical anchor having a sharp end for piercing said bone tissue in an articular surface of at least one of said sacrum and said ilium.

89. The method of claim 88, wherein said fusion implant comprises a body having two helical anchors for engaging with said bone tissue, and a cavity is formed between said two helical anchors.

90. The method of claim 89, wherein said two helical anchors are arranged in an interwoven double helix pattern, and each helical anchor has a sharp end for piercing said bone tissue.

91. The method of claim 67, wherein said joint fusion implant comprises a cavity for holding a bone fusion-promoting material to promote fusion of said targeted joint.

92. The method of claim 67, wherein said first incision is positioned about between a posterior inferior iliac spine and a sacrum of said sacroiliac joint.

93. The method of claim 67, further comprising:
a. creating a second incision in the patient's skin proximal to said sacroiliac joint;
b. inserting a second cannula into said second incision;
c. creating a second void in said sacroiliac joint;
d. passing a second washer device through said second cannula for placement at a superficial aspect of said targeted joint within said second incision; and
e. inserting a second joint fusion implant into said void through said second washer device.

94. The method of claim 93, wherein said second incision is positioned about between a posterior superior iliac spine and a superior lateral aspect of the sacrum of said sacroiliac joint.

95. A method for repairing a targeted joint of a patient, comprising:
a. creating an incision in the patient's skin proximal to said targeted joint;
b. inserting a washer device through said incision and into a superficial aspect of said targeted joint; and
c. inserting a joint fusion implant into said void through said washer device.

96. The method of claim 95, further comprising inserting a cannula into said incision.

97. The method of claim 96, further comprising creating a void in said joint prior to inserting said joint fusion device, wherein a drill is inserted through said cannula and into said incision, and drills said void through tissues in said targeted joint

97. The method of claim 97, wherein the drill comprises a drill bit having an outer diameter that is substantially equal to an outer diameter of a ring structure of said washer device, wherein the washer device is operable to be inserted in shallow portion of said void.

95. The method of claim 95, wherein said washer device comprises guidance tabs attached to said washer device for guiding the insertion of said joint fusion implant into said targeted joint.

100. The method of claim 99, wherein said guidance tabs are fixedly attached to said washer with break-away connections that allow the guidance tabs to be removed from the washer device once said washer device is engaged with said targeted joint.

101. The method of claim 100, further comprising coupling a washer driver to said guidance tabs of said washer device prior to the insertion of said washer device into said targeted joint.

102. The method of claim 101, wherein said washer device and said washer driver are passed through said cannula when said washer device is inserted into said targeted joint.

103. The method of claim 102, further comprising removing said washer driver from said washer device, leaving said washer device engaged with said targeted joint.

104. The method of claim 103, wherein inserting a joint fusion implant into said void comprises using an implant inserter to pass said fusion implant through said washer, wherein said inserter moves said implant through said cannula in a linear, co-axial path through the cannula until said at least one fixation element reaches said washer device, and rotating said joint fusion implant into said void such that an at least one fixation element of said fusion implant engages with at least one articular surface of said joint.

105. The method of claim 104, further comprising removing said guidance tabs from said ring structure of said washer device after said fusion implant is inserted into said void.

106. The method of claim 105, wherein said guidance tabs are fixedly attached to said washer with break-away connections that allow the guidance tabs to be removed from the washer device once said washer device is engaged with said targeted joint.

107. The method of claim 95, further comprising inserting a guide wire into said sacroiliac joint of the patient, slotting a joint cutting tool over said guide wire, and placing a cannula over said guide wire and said joint cutting tool, such that said guide wire and joint cutting tool are within a barrel of said cannula and an end of said cannula is within said targeted joint.

108. The method of claim 95, further comprising removing cortical tissue from said articular surfaces of said articulating bones prior to inserting said fusion implant into said void.

109. The method of claim 95, wherein said washer device has a washer ring having at least one washer fixation element for engagement with said targeted joint.

110. The method of claim 109, wherein said washer fixation element includes at least one distal tang that is operable to be inserted into the targeted joint and is operable to stabilize medical instrumentation for inserting said joint fusion implant.

111. The method of claim 95, wherein said fusion implant has at least one fixation element for engagement with bone tissue in an articular surface of at least one of the articulating bones of said targeted joint.

112. The method of claim 111, further comprising rotating said at least one fixation element to engage said bone tissue in at least one articular surface of said targeted joint.

113. The method of claim 112, wherein said at least one implant fixation element is operable to engage bone tissue in articular surfaces of two articulating bones in said joint to fix said articular surfaces in position relative to each other.

114. The method of claim 112, wherein said at least one implant fixation element includes at least one secondary fixation element thereon.

115. The method of claim 114, wherein said at least one secondary fixation element includes a protrusion on an outer edge of said at least one implant fixation element operable to engage with an articular surface of said targeted joint.

116. The method of claim 114, wherein said at least one secondary fixation element includes threading on an outer edge of said at least one implant fixation element.

117. The method of claim 114, wherein said at least one secondary fixation element includes fluting on an outer edge of said at least one implant fixation element.

118. The method of claim 111, wherein said at least one fixation element comprises a helical anchor having a sharp end for piercing said bone tissue in an articular surface of at least one of said sacrum and said ilium.

119. The method of claim 111, wherein said fusion implant comprises a body having two helical anchors for engaging with said bone tissue, and a cavity is formed between said two helical anchors.

120. The method of claim 119, wherein said two helical anchors are arranged in an interwoven double helix pattern, and each helical anchor has a sharp end for piercing said bone tissue.

121. The method of claim 95, wherein said joint fusion implant comprises a cavity for holding a bone fusion-promoting material to promote fusion of said targeted joint.

122. The method of claim 95, wherein said first incision is positioned about between a posterior inferior iliac spine and a sacrum of said sacroiliac joint.

123. The method of claim 95, further comprising:
a. creating a second incision in the patient's skin proximal to said sacroiliac joint;
b. inserting a second cannula into said second incision;
c. creating a second void in said sacroiliac joint;
d. passing a second washer device through said second cannula for placement at a superficial aspect of said targeted joint within said second incision; and
e. inserting a second joint fusion implant into said void through said second washer device.

124. The method of claim 123, wherein said second incision is positioned about between a posterior superior iliac spine and a superior lateral aspect of the sacrum of said sacroiliac joint.
